SEQUENCE LISTING (22 sequences)

SEQ ID NO: 1 - protein, 500 aa, Euphorbia lagascae
  MISC_FEATURE: NCBI Accession No. AAL62063.1
MEQKNLSFPSILISFLLVLILVVVMRLWKKQNPPPGPWKFPIIGNLPHLLLTSDLGHERFRALAQIYGPVMSLQIGQVSAVVISSAEAAKEVMKTQADAFAQRPIVLDAQIVFYNRKDVLFASYGDHWRQMKKIWILEFLSAKKVQSSRLIREEEMEDAITFLRSKAGSPVNITKIIYGIIISIMIRTSVGNCKQKERLLSVADAVNEAATSFGTADAFPTWKLLHYIIGAESKPRRLHQEIDDILEEILNEHKANKPFEADNLMDVLLNLQKNGNVPVPVTNESIKASVLQMFTAGSETTSKATEWVMAELMKNPTELRKAQEEVRQVFGEMGKVDESRFHDLKFFKLVVKETLRLHPPVVLIPRECRETTRIDGYEIHPNTRIVVNAWAIGRDPNTWSEPGKFNPERFKDCAIDYKGTTFELVPFGAGKRICPGITSAITNLEYVIINLLYHFNWELADGITPQTLDMTEAIGGALRKKIDLKLIPIPYQVSLGSNIS

SEQ ID NO: 2 - DNA, 1784 bp, Glycine max
  misc_feature: clone sfl1.pk0045.g7
gcacgagtttagttcttttccatggctctattattcttctactttttggttcttatttcctttgccttcacaaccattatagtacaaaaaataagaaagaaacccaagaaaactgatgacacaacttgtaaaataccccatggtcctagaaagctacctattataggaaatatatacaatctgctatgctctcaaccccatagaaaattaagagacctggccataaaatatggacccgtgatgcatcttcaacttggacaggtttcaactattgtcatttcatcccctgagtgtgctagggaagtgatgaaaacccatgacattaactttgccacaaggcctaaagttctagctattgaaataatgtcttacaattccacaagtatagcttttgctggctatggaaattattggaggcagctaagaaaaatatgcacattggagcttttaagcctaaaacgcgtcaactcattccagccaattagagaagatgagctcttcaatcttgtcaaatggattgattcaaagaaaggatcccccatcaacctcactgaagcagtacttacatcaatttacacaattgcttcaagggctgcctttggcaagaattgcaaagaccaagaaaaatttatatcagtggttaaaaaaacatcaaaacttgctgcaggttttggcattgaagatttgtttccttctgttacttggcttcaacatgtcactggcttgagggctaagcttgagaggttgcatcaacagactgatcagataatggaaaacatcatcaatgagcataaagaggcaaattcgaaagccaaggatgaccaaagtgaagcagaagaagatcttgtggatgttctcatacaatatgaggatgggagcaagaaggatttttccttaactagaaacaagatcaaggccataattctggacatttttgctgctggaggtgagacaacagcaacaaccatagattgggcaatggctgaaatggtaaagaatccaacagtaatgaagaaagcacaatctgaggtgagagaggtatgcaatatgaaagcaagggttgatgaaaattgcatcaatgaacttcaatatttgaaactaattgtgaaagagaccttgaggttgcaccctccagctcctcttttgctgccaagagaatgtggtcaaacatgtgagatacatggttatcacataccagccaaaaccaaggtcatagtcaatgcctgggcaattggaagagatccaaactattggactgaatcagagaggttttatcctgagaggttcattgatagcactattgactacaaagggagtaattttgagttcattccttttggtgctggaagaaggatatgcgcaggaagcacatttgctttgagagctgcggaactagcccttgcaatgttgttgtatcactttgattggaagcttccaagtggaatgagaagtggagaactggacatgagtgaggattttggagtcacaactataagaaaagataatctattcttggttccttttccatatcatcctttgcctgtctcatgagctcagaatgagaaactgaaacatttggtgatggctgtcacatttacttcagaataaagatcttctgttaattgaaattagactttgtgctgtgattggttgtaacagaagatggaaattgtgactactgcatattaaaatagcttttatatatcctcaatgttaattaactttgataaaaaaaaaaaaaaaaaaaaaaaaaaaaaaaaaaaaaaaaaaaaaaaaaaaaaaaa

SEQ ID NO: 3 - protein, 509 aa, Glycine max
  MISC_FEATURE: translation of clone sfl1.pk0045.g7
MALLFFYFLVLISFAFTTIIVQKIRKKPKKTDDTTCKIPHGPRKLPIIGNIYNLLCSQPHRKLRDLAIKYGPVMHLQLGQVSTIVISSPECAREVMKTHDINFATRPKVLAIEIMSYNSTSIAFAGYGNYWRQLRKICTLELLSLKRVNSFQPIREDELFNLVKWIDSKKGSPINLTEAVLTSIYTIASRAAFGKNCKDQEKFISVVKKTSKLAAGFGIEDLFPSVTWLQHVTGLRAKLERLHQQTDQIMENIINEHKEANSKAKDDQSEAEEDLVDVLIQYEDGSKKDFSLTRNKIKAIILDIFAAGGETTATTIDWAMAEMVKNPTVMKKAQSEVREVCNMKARVDENCINELQYLKLIVKETLRLHPPAPLLLPRECGQTCEIHGYHIPAKTKVIVNAWAIGRDPNYWTESERFYPERFIDSTIDYKGSNFEFIPFGAGRRICAGSTFALRAAELALAMLLYHFDWKLPSGMRSGELDMSEDFGVTTIRKDNLFLVPFPYHPLPVS

SEQ ID NO: 4 - DNA, 36 bp, Artificial Sequence
  Oligonucleotide primer PSO355954 sense-3
gcggccgcatggctctattattcttctactttttgg

SEQ ID NO: 5 - DNA, 35 bp, Artificial Sequence
  Oligonucleotide primer PSO355954 anti-sense-3
gcggccgctcatgagacaggcaaaggatgatatgg

SEQ ID NO: 6 - DNA, 35 bp, Artificial Sequence
  Oligonucleotide primer gene 2 overexpress sense
gcggccgcatggctctattattcttctactttttg

SEQ ID NO: 7 - DNA, 35 bp, Artificial Sequence; identical to SEQ ID NO: 5
  Oligonucleotide primer gene 2 overexpress antisense

SEQ ID NO: 8 - DNA, 1952 bp, Glycine max
  misc_feature: P450 genomic sequence
gcccttgcggccgcatggctctattattcttctactttttggttcttatttcctttgccttcacaaccattatagtacaaaaaataagaaagaaacccaagaaaactgatgacacaacttgtaaaataccccatggtcctagaaagctacctattataggaaatatatacaatctgctatgctctcaaccccatagaaaattaagagacctggccataaaatatggacccgtgatgcatcttcaacttggacaggtttcaactattgtcatttcatcccctgagtgtgctagggaagtgatgaaaacccatgacattaactttgccacaaggcctaaagttctagctattgaaataatgtcttacaattccacaagtatagcttttgctggctatggaaattattggaggcagctaagaaaaatatgcacattggagcttttaagcctaaaacgcgtcaactcattccagccaattagagaagatgagctcttcaatcttgtcaaatggattgattcaaagaaaggatcccccatcaacctcactgaagcagtacttacatcaatttacacaattgcttcaagggctgcctttggcaagaattgcaaagaccaagaaaaatttatatcagtggttaaaaaaacatcaaaacttgctgcaggttttggcattgaagatttgtttccttctgttacttggcttcaacatgtcactggcttgagggctaagcttgagaggttgcatcaacaggctgatcagataatggaaaacatcatcaatgagcataaagaggcaaattcgaaagccaaggatgaccaaagtgaagcagaagaagatcttgtggatgttctcatacaatatgaggatgggagcaagaaggatttttccttaactagaaacaagatcaaggccataattctggtatggatatatcataacctcttggatatacattttatgaacataatcttttcagacactgtctgctagttatttctgtttgttggttttaaacacttgttttggaaacaatcttccaaacttaatagattttggatgaggaattgattgctgaataacataaaattatttttaaaaaaatagttttaaaataaaaaagtgagaagagaatcaaacggagatcctaaatgttcaacaattgccccctttatatcttatttcccactagtgagatctttgaagtagtgaaatatttgccacatattccttgcagcaagtaggttttctttctgttttaactatctctttggacatccttgcactatcttaatcttatcggttaattatttttcaggacatttttgctgctggaggtgagacaacagcaacaaccatagattgggcaatggctgaaatggtaaagaatccaacagtaatgaagaaagcacaatctgaggtgagagaggtatgcaatatgaaagcaagggttgatgaaaattgcatcaatgaacttcaatatttgaaactaattgtgaaagagaccttgaggttgcaccctccagctcctcttttgctgccaagagaatgtggtcaaacatgtgagatacatggttatcacataccagccaaaaccaaggtcatagtcaatgcctgggcaattggaagagatccaaactattggactgaatcagagaggttttatcctgagaggttcattgatagcactattgactacaaagggagtaattttgagttcattccttttggtgctggaagaaggatatgcgcaggaagcacatttgctttgagagctgcggaactagcccttgcaatgttgttgtatcactttgattggaagcttccaagtggaatgagaagtggagaactggacatgagtgaggattttggagtcacaactataagaaaagataatctattcttggttccttttccatatcatcctttgcctgtctcatgagcggccgcaagggc

SEQ ID NO: 9 - DNA, 6383 bp, Artificial Sequence
  6383 bp fragment from pKS231
cgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 10 - protein, 656 aa, Artificial Sequence
  mutant soybean ALS
MPHNTMAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVARRMIGTDAFQETPIVEVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQLEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 11 - DNA, 8966 bp, Artificial Sequence
  Plasmid pKS210
gatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcg

SEQ ID NO: 12 - DNA, 40 bp, Artificial Sequence
  Oligonucleotide primer KS210-Kpn-Hyg-sense
gccggggtaccggcgcgcccgatcatccggatatagttcc

SEQ ID NO: 13 - DNA, 40 bp, Artificial Sequence
  Oligonucleotide primer KS210-Kpn-Hyg-antisense
gccggggtaccggcgcgccgttctatagtgtcacctaatc

SEQ ID NO: 14 - DNA, 8911 bp, Artificial Sequence
  Plasmid pDN10
cggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 15 - DNA, 43 bp, Artificial Sequence
  Oligonucleotide primer gene 2 hairpin 2
caaagaccaagaaaaatttatacaacctcaaggtctctttcac

SEQ ID NO: 16 - DNA, 42 bp, Artificial Sequence
  Oligonucleotide primer gene 2 hairpin 3
tgaaagagaccttgaggttgtataaatttttcttggtctttg

SEQ ID NO: 17 - DNA, 7935 bp, Artificial Sequence
  Recombinant DNA fragment PHP23366A
cgcgccggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgctcatgagacaggcaaaggatgatatggaaaaggaaccaagaatagattatcttttcttatagttgtgactccaaaatcctcactcatgtccagttctccacttctcattccacttggaagcttccaatcaaagtgatacaacaacattgcaagggctagttccgcagctctcaaagcaaatgtgcttcctgcgcatatccttcttccagcaccaaaaggaatgaactcaaaattactccctttgtagtcaatagtgctatcaatgaacctctcaggataaaacctctctgattcagtccaatagtttggatctcttccaattgcccaggcattgactatgaccttggttttggctggtatgtgataaccatgtatctcacatgtttgaccacattctcttggcagcaaaagaggagctggagggtgcaacctcaaggtctctttcacaattagtttcaaatattgaagttcattgatgcaattttcatcaacccttgctttcatattgcatacctctctcacctcagattgtgctttcttcattactgttggattctttaccatttcagccattgcccaatctatggttgttgctgttgtctcacctccagcagcaaaaatgtccagaattatggccttgatcttgtttctagttaaggaaaaatccttcttgctcccatcctcatattgtatgagaacatccacaagatcttcttctgcttcactttggtcatccttggctttcgaatttgcctctttatgctcattgatgatgttttccattatctgatcagtctgttgatgcaacctctcaagcttagccctcaagccagtgacatgttgaagccaagtaacagaaggaaacaaatcttcaatgccaaaacctgcagcaagttttgatgtttttttaaccactgatataaatttttcttggtctttgcaattcttgccaaaggcagcccttgaagcaattgtgtaaattgatgtaagtactgcttcagtgaggttgatgggggatcctttctttgaatcaatccatttgacaagattgaagagctcatcttctctaattggctggaatgagttgacgcgttttaggcttaaaagctccaatgtgcatatttttcttagctgcctccaataatttccatagccagcaaaagctatacttgtggaattgtaagacattatttcaatagctagaactttaggccttgtggcaaagttaatgtcatgggttttcatcacttccctagcacactcaggggatgaaatgacaatagttgaaacctgtccaagttgaagatgcatcacgggtccatattttatggccaggtctcttaattttctatggggttgagagcatagcagattgtatatatttcctataataggtagctttctaggaccatggggtattttacaagttgtgtcatcagttttcttgggtttctttcttattttttgtactataatggttgtgaaggcaaaggaaataagaaccaaaaagtagaagaataatagagccatgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtaccgg

SEQ ID NO: 18 - DNA, 8120 bp, Artificial Sequence
  Recombinant DNA construct PHP23367A
gcgccggtacccgggtctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccccaccgcggtggcggcccgcgccaagcttggatcctcgaagagaagggttaataacacacttttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccgcatggctctattattcttctactttttggttcttatttcctttgccttcacaaccattatagtacaaaaaataagaaagaaacccaagaaaactgatgacacaacttgtaaaataccccatggtcctagaaagctacctattataggaaatatatacaatctgctatgctctcaaccccatagaaaattaagagacctggccataaaatatggacccgtgatgcatcttcaacttggacaggtttcaactattgtcatttcatcccctgagtgtgctagggaagtgatgaaaacccatgacattaactttgccacaaggcctaaagttctagctattgaaataatgtcttacaattccacaagtatagcttttgctggctatggaaattattggaggcagctaagaaaaatatgcacattggagcttttaagcctaaaacgcgtcaactcattccagccaattagagaagatgagctcttcaatcttgtcaaatggattgattcaaagaaaggatcccccatcaacctcactgaagcagtacttacatcaatttacacaattgcttcaagggctgcctttggcaagaattgcaaagaccaagaaaaatttatatcagtggttaaaaaaacatcaaaacttgctgcaggttttggcattgaagatttgtttccttctgttacttggcttcaacatgtcactggcttgagggctaagcttgagaggttgcatcaacagactgatcagataatggaaaacatcatcaatgagcataaagaggcaaattcgaaagccaaggatgaccaaagtgaagcagaagaagatcttgtggatgttctcatacaatatgaggatgggagcaagaaggatttttccttaactagaaacaagatcaaggccataattctggacatttttgctgctggaggtgagacaacagcaacaaccatagattgggcaatggctgaaatggtaaagaatccaacagtaatgaagaaagcacaatctgaggtgagagaggtatgcaatatgaaagcaagggttgatgaaaattgcatcaatgaacttcaatatttgaaactaattgtgaaagagaccttgaggttgtataaatttttcttggtctttgcaattcttgccaaaggcagcccttgaagcaattgtgtaaattgatgtaagtactgcttcagtgaggttgatgggggatcctttctttgaatcaatccatttgacaagattgaagagctcatcttctctaattggctggaatgagttgacgcgttttaggcttaaaagctccaatgtgcatatttttcttagctgcctccaataatttccatagccagcaaaagctatacttgtggaattgtaagacattatttcaatagctagaactttaggccttgtggcaaagttaatgtcatgggttttcatcacttccctagcacactcaggggatgaaatgacaatagttgaaacctgtccaagttgaagatgcatcacgggtccatattttatggccaggtctcttaattttctatggggttgagagcatagcagattgtatatatttcctataataggtagctttctaggaccatggggtattttacaagttgtgtcatcagttttcttgggtttctttcttattttttgtactataatggttgtgaaggcaaaggaaataagaaccaaaaagtagaagaataatagagccatgcggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgggccgctctagaactagtggatcccccgggctgcaggaattcgatatcaagcttatcgataccgtcgacctcgagggggggcccggtaccgg

SEQ ID NO: 19 - DNA, 26 bp, Artificial Sequence
  gene specific primer 1
tggccaggtctcttaattttctatgg

SEQ ID NO: 20 - DNA, 25 bp, Artificial Sequence
  gene specific primer 2
aggaccatggggtattttacaagtt

SEQ ID NO: 21 - DNA, 2779 bp, Glycine max
  misc_feature (1)..(2779): Promoter region
ccttgggggtcaccgggtttgagtgctcctttaagcctatgctgatctcatatggttggagcattctcgcaaaacatcgtgaccatgactggtctccctatgatcttacttagtgagagtgacctgacaaacccattgtgtagtgtgtcttgttatgtactcctaagcgccccagggtagtttttcactgacatggtaccacattgcatatagaattgagtcttagcataactattgcatatgcttgctaattgatgtgttattatatcttgatcgaagtgtgggattcttgtgtaatgtgattgataattgaaaagtgaattttgaatgacgaaatggtgaagttacgtgagctatgtttaagcaagtggtatctcatttatataatatgtatatttaattgtcttgtttctctattagctaggaatgtgataactcactccctgtgtgttgttgtgtttgatcctgtgatgatcttgaacttgtgttcgggggagcagatgaataggtggatgactatgaagaacctcatgctagaggacacaggaacaccatgctctgataggatgtgacattaggatataggttctatattaattgtatgagacttatatgactttctttgagtcgagatgactttattatttatttggacaagtttgaatatgatgtagaagaaagtgaatgtgagccttttacccatttgaaaagcttgtatttaaaaatgttttaaaaatacttttaattaatatttgaatttttattcctttattaatatatatgtgaggggtagagggtgtcacaataactactgtactttggacagtatttgggtctaaacaatcaatattttcgctcttcattcgtaactcaagaatcaatgcagaacaatatatttgtatttatgggctgaattatctatatataaataaaagaaaagagtagtaagaaaaaaaagatatatttttgaggttatgtctgttcgtaaaataactttaataactatatttctaaaaataaccgattaaaaagggatatgtgaaaatataatattttatattacattctattgggtaaagaaatatatattgatgaattatacaagattattatttaaacaaaatttataataaattataaggtaaggataatataattacttttttggtataaattttgttatgttgtgagtaatattgaagaagagagaaggaaaagagaagtgataaacaagacaaaaacgatccccttcctccaaatcatcaattggagctgaggtggcagcgtaatagtaaaaagttaaacttttatatattataaatataaatagattgagatttttaattttaaaaaacaaattgagattttcaagacaagcaaagttgtttgtatatatttgacctgttatggtaaaaatggtaaaacttcaactacaatctctgttacgcattaagcaaattttgggattttcaatgaaagctaagttgtttgtagttgtactgacctgttaggcaaagttgtttgtactgacctgttgtggtaaaattggcaaaacttcatctacaatctctgttagtgataatatcaaagtaaaaaaaaaaaaaatgttaattatagaagtaaggatagaaaatttgaggattttaagtagaagagaattgatggaaagatcaattttgactaattttaaaatagaggaacataagtagctaaaaaaaataatttggaactaaatactgtaaaaatatttacatggaagtgtaactaataaaataataatttaattgaaaaacactgtgatagtggaaaggtttttttatggatttttatacaactaaaaaattgtagacttttatagtaatttttaaaaaaatcatatctaaagtaatttataattaaaaatacttttaaaattgttatttaatatatagtgtgatttctattgattactattgtgtgaagttttacactagcaatatcaaaattaaacccataaaataataacttagttggaacatattaacaccataaaagtttttacaagttatcttattatagttggcacaactttaccgctgtatcagggctcgccctctatcttattatagtttattatattaaaaaatgctgacttttataatagttattttacaagtaagaagatttacaattaatgcatactattaaagatgatgatagtcaatcaaaattaaacttataaaacagaaaactttacattaacttccgatacgttatttgacaccagaaaaaaaaaagtattagaatgatgtttaaaataaggagatgtttgtgtatgattactctataaaataagatgctgccagtacaaggataatgtttaagtcatgaaaatgaccatttgtcctcttttgaaataaagatagcctttttattgagctaaactgaactatttttcaatttaaagtagcaaaagattccatagaaaagaaatcaaacacggaacaaaacattctaaatataggagaatctgaagatgatgatatatagtgttctaaccaaaaagaaaagttaacaagtaatcaccaagcaaaatactgagccatgtgtatataaatagacatcagtaggatcagacttgtattacaactccgctactttttagttcttttccatggctctattattcttctactttttggttcttatttcctttgccttcacaaccattatagtacaaaaaataagaaagaaacccaagaaaactgatgacacaacttgtaaaataccccatggtcct

SEQ ID NO: 22 - protein, 509 aa, Glycine max
  MISC_FEATURE (1)..(509): Translation of coding sequence in genomic fragment
MALLFFYFLVLISFAFTTIIVQKIRKKPKKTDDTTCKIPHGPRKLPIIGNIYNLLCSQPHRKLRDLAIKYGPVMHLQLGQVSTIVISSPECAREVMKTHDINFATRPKVLAIEIMSYNSTSIAFAGYGNYWRQLRKICTLELLSLKRVNSFQPIREDELFNLVKWIDSKKGSPINLTEAVLTSIYTIASRAAFGKNCKDQEKFISVVKKTSKLAAGFGIEDLFPSVTWLQHVTGLRAKLERLHQQADQIMENIINEHKEANSKAKDDQSEAEEDLVDVLIQYEDGSKKDFSLTRNKIKAIILDIFAAGGETTATTIDWAMAEMVKNPTVMKKAQSEVREVCNMKARVDENCINELQYLKLIVKETLRLHPPAPLLLPRECGQTCEIHGYHIPAKTKVIVNAWAIGRDPNYWTESERFYPERFIDSTIDYKGSNFEFIPFGAGRRICAGSTFALRAAELALAMLLYHFDWKLPSGMRSGELDMSEDFGVTTIRKDNLFLVPFPYHPLPVS

(19) United States
(12) Patent Application Publication
(10) Pub. No.: US 2006/0156430 A1
(43) Pub. Date: Jul. 13, 2006

(54) Novel cytochrome P450 monooxygenase

(76) Inventors: Brian McGonigle, Wilmington, DE (US); Saverio Carl Falco, Wilmington, DE (US); John D. Everard, Wilmington, DE (US); Nicole A. Swinson, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/329,255

(22) Filed: Jan. 10, 2006

Related U.S. Application Data
(60) Provisional application No. 60/643,438, filed on Jan. 13, 2005.
(60) Provisional application No. 60/661,980, filed on Mar. 15, 2005.

(51) Int. Cl. A01H 5/00; C12N 5/04; A23K 1/00; C12N 15/82; A01H 1/00; C12P 7/16

(52) U.S. Cl. 800/278; 800/312; 435/415; 435/468; 426/635; 554/8; 435/160

(57) ABSTRACT

This invention relates to an isolated polynucleotide encoding a cytochrome P450 monooxygenase that in the presence of linoleic acid results in production of 1-octen-3-ol. The invention also relates to the construction of a recombinant DNA construct comprising all or a portion of the polynucleotide encoding the cytochrome P450 monooxygenases of the invention, in sense or antisense orientation, wherein expression of the recombinant DNA construct results in production of altered levels 1-octen-3-ol in a transformed host cell.

This application claims the benefit of U.S. Provisional Application No. 60/643,438, filed Jan. 13, 2005, and U.S. Provisional Application No. 60/661,980, filed Mar. 15, 2005, each of which is hereby incorporated by reference in its entirety.

### FIELD OF THE INVENTION

This invention is in the field of plant molecular biology. More specifically, this invention pertains to nucleic acids encoding a novel cytochrome P450 monooxygenase that is believed useful in the biosynthesis of 1-octen-3-ol in the presence of linoleic acid. The invention also pertains to plants and plant parts having altered levels of 1-octen-3-ol.

### BACKGROUND OF THE INVENTION

Lipids are a major source of flavor compounds responsible for desirable and undesirable characteristics in foods. They are believed to be responsible for the desirable flavor of some cheeses and fresh milk as well as the characteristic flavor of mushrooms, green beans, tomatoes, cucumbers and ripe fruit. In soybeans they are thought to be at least partially responsible for the “beany” and “grassy” off-flavors that limit the potential for wider use of this economical and healthy source of protein. Enzymatically-catalyzed degradation of fatty acids produces aroma compounds including eight carbon compounds such as 1-octanol, 3(R)-octanol, 3-octanone, (R)-1-octen-3-ol, and 2(Z)-octen-1-ol. These eight carbon compounds are thought to be the main contributors to the characteristic flavor of mushroom.

1-octen-3-ol has a forest, earthy-mushroom aroma. It is found naturally in banana, beans, beef, beer, hyacinth, leek, licorice, lily, malt, milk, mushroom, peppermint oil, pork, pumpkin, rice, shrimp, soybean, spearmint oil, thyme, and tomato, among others. 1-octen-3-ol is also found in milk, butter, and cheeses. Natural 1-octen-3-ol is an expensive compound used in the flavor industry. It is commonly produced from extracts obtained from mushrooms.

Chemical oxidation of 1-octen-3-ol produces 1-octen-3-one. This is also a flavor compound with an earthy-mushroom type odor (Schwab, W. and Schreier, P, 2002, Enzymatic formation of flavor volatiles, Chapter 15 in Lipid Biotechnology, T. M. Kuo and H. M. Gardener, eds). The odor potency of 1-octen-3-one is 200 times greater than that of 1-octen-3-ol and 1-octen-3-one is a principal odorant in soy products.

The enzyme or enzymes that catalyze production of 1-octen-3-ol have yet to be identified. It is known that breakdown of linoleic acid in mushrooms produces 1-octen-3-ol and 10-oxo-trans-decenoic acid (ODA). Until now a lipoxygenase and a hydroperoxide lyase were thought to be responsible for the production of (R)-1-octen-3-ol in mushrooms (Husson, F. et al., 2001, Process Biochem. 37:177-182). In the present application, soybean polynucleotides encoding novel cytochrome P450 monooxygenases are shown to produce 1-octen-3-ol from linoleic acid.

### SUMMARY OF THE INVENTION

The present invention concerns an isolated polynucleotide comprising a nucleotide sequence encoding a cytochrome P450 polypeptide having an amino acid sequence of at least 80% sequence identity, based on the Clustal V method of alignment, when compared to an amino acid sequence of SEQ ID NO:3, wherein expression of the polypeptide in an appropriate host cell transformed with the isolated polynucleotide, in the presence of linoleic acid, results in an increased level of 1-octen-3-ol in the transformed host cell when compared to an nontransformed host cell; or a complement of the nucleotide sequence, wherein the complement and the nucleotide sequence consist of the same number of nucleotides and are 100% complementary.

An embodiment of the present invention is a portion of the above-identified polynucleotide which is capable of suppressing production of 1-octen-3-ol when introduced into cells that normally produce 1-octen-3-ol.

An embodiment of the present invention is an isolated polynucleotide comprising SEQ ID NO: 2 or a portion thereof wherein the portion is capable of suppressing production of 1-octen-3-ol when introduced into cells that normally produce 1-octen-3-ol.

The nucleotide sequence of the present invention may further comprise a native cytochrome P450 promoter region, a native cytochrome P450 terminator region, and a native cytochrome P450 intron.

An embodiment of the present invention is a recombinant DNA construct comprising the above-identified polynucleotide and a transgenic cell comprising the recombinant DNA construct of the present invention. The recombinant construct of the present invention may result in an altered level of 1-octen-3-ol, wherein the altered level may be an increased or a decreased level.

Other embodiments of the present invention include a plant and a seed comprising the recombinant DNA construct. The present invention includes a soybean plant and a soybean seed. The soybean plant whose genome may comprise a disruption of the polynucleotide of the present invention, wherein the disruption results in a plant exhibiting reduced 1-octen-3-ol when compared to its wild type counterpart is an embodiment of the present invention.

An embodiment of the present invention is a method for transforming a cell, comprising transforming a cell with the recombinant DNA construct of the present invention. The methods of the present invention include producing a plant comprising transforming a plant cell with the recombinant DNA construct, regenerating a plant from the transformed plant cell; and growing the transformed plant under conditions suitable for the expression of the recombinant DNA construct, the grown transformed plant having an altered level of 1-octen-3-ol when compared to a non-transformed plant.

A method of producing 1-octen-3-ol comprising transforming a host cell with the recombinant DNA construct and adding linoleic acid to the transformed host cell in an amount sufficient for said host cell to produce 1-octen-3-ol, is an embodiment of the present invention. The host cell is selected from the group consisting of yeast and an insect cell.

Other embodiments of the present invention include soybean grain, soybean protein product, soybean oil, feed, and food.

### BRIEF DESCRIPTION OF THE SEQUENCE LISTING

The invention can be more fully understood from the following detailed description and the accompanying Sequence Listing that forms part of this application.

The sequence descriptions and Sequence Listing attached hereto comply with the rules governing nucleotide and/or amino acid sequence disclosures in patent applications as set forth in 37 C.F.R. §1.821-1.825.

SEQ ID NO:1 is the amino acid sequence of the Euphorbia lagascae cytochrome P450 monooxygenase (CYP726A1) that catalyzes conversion of linoleic acid to vernolic acid and has NCBI Accession No. AAL62063.1.

SEQ ID NO:2 is the nucleotide sequence of the entire cDNA insert in clone sfl1.pk0045.g7 encoding a cytochrome P450 monooxygenase.

SEQ ID NO:3 is the deduced amino acid sequence obtained from translating nucleotides 22 through 1548 of SEQ ID NO:2.

SEQ ID NO:4 is the nucleotide sequence of oligonucleotide primer PSO355954 sense-3 used to amplify DNA from clone sfl1.pk0045.g7.

SEQ ID NO:5 is the nucleotide sequence of oligonucleotide primer PSO355954 anti-sense-3 used to amplify DNA from clone sfl1.pk0045.g7.

SEQ ID NO:6 is the nucleotide sequence of oligonucleotide primer gene 2 overexpress sense used to amplify genomic DNA and the polynucleotide corresponding to the open reading frame in clone sfl1.pk0045.g7.

SEQ ID NO:7 is the nucleotide sequence of oligonucleotide primer gene 2 overexpress antisense used to amplify genomic DNA and the polynucleotide corresponding to the open reading frame in clone sfl1.pk0045.g7.

SEQ ID NO:8 is the nucleotide sequence of the genomic DNA fragment encoding a cytochrome P450 that results in production of 1-octen-3-ol.

SEQ ID NO:9 is the nucleotide sequence of the 6383 polynucleotide fragment obtained by Kpn I digestion of pKS231 and comprising a plant selectable marker gene cassette comprising a promoter operably linked to a mutant soybean ALS gene and the soybean ALS 3′ transcription terminator, and a gene expression cassette comprising the KTi3 promoter, a unique Not I restriction endonuclease site, and the Kti3 terminator region.

SEQ ID NO:10 is the deduced amino acid sequence of the herbicide resistant mutant soybean ALS present in the 6383 nucleotide fragment of SEQ ID NO:19.

SEQ ID NO:11 is the nucleotide sequence of plasmid pKS210.

SEQ ID NO:12 is the nucleotide sequence of oligonucleotide primer KS210-Kpn-Hyg-sense used to amplify the bacterial origin of replication and the HPT gene from plasmid pKS210.

SEQ ID NO:13 is the nucleotide sequence of oligonucleotide primer KS210-Kpn-Hyg-antisense used to amplify the bacterial origin of replication and the HPT gene from plasmid pKS210.

SEQ ID NO:14 is the nucleotide sequence of plasmid pDN10.

SEQ ID NO:15 is the nucleotide sequence of oligonucleotide primer gene 2 hairpin 2 used to amplify a portion of the cDNA insert in clone sfl1.pk0045.g7.

SEQ ID NO:16 is the nucleotide sequence of oligonucleotide primer gene 2 hairpin 3 used to amplify a portion of the cDNA insert in clone sfl1.pk0045.g7.

SEQ ID NO:17 is the nucleotide sequence of the Asc I recombinant DNA fragment PHP23366A.

SEQ ID NO:18 is the nucleotide sequence of the Asc I recombinant DNA fragment called PHP23367A.

SEQ ID NO:19 is the nucleotide sequence of oligonucleotide primer gene specific primer 1 used in amplifying the promoter region of a soybean P450 monooxygenase.

SEQ ID NO:20 is the nucleotide sequence of oligonucleotide primer gene specific primer 2 used in amplifying the promoter region of a soybean P450 monooxygenase.

SEQ ID NO:21 is the nucleotide sequence of the promoter region of a soybean P450 monooxygenase.

SEQ ID NO:22 is the amino acid sequence deduced from the coding sequence in the genomic fragment of SEQ ID NO:8.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

In the context of this disclosure, a number of terms shall be utilized. The terms “polynucleotide,” “isolated polynucleotide,” “nucleic acid fragment,” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. An isolated polynucleotide of the present invention may include all or part of the isolated polynucleotide, such as, for example, a polynucleotide comprising the nucleotide sequence of SEQ ID NO: 2, a portion thereof, or the full complement of such nucleotide sequence.

The term “isolated” refers to materials, such as “isolated polynucleotide” and/or “isolated polypeptides,” which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The present invention is directed to an isolated polynucleotide encoding a cytochrome P450 monooxygenase that, in the presence of linoleic acid, result in production of 1-octen-3-ol.

The terms “cytochrome P450 monooxygenase,” “cytochrome P450,” “P450” and “CYP450” are used interchangeably herein. They comprise a large number of polypeptides that are grouped into families based on sequence homology. Cytochrome P450 monooxygenases are involved in the biosynthesis of a multitude of compounds, as described in Nelson D. R., 1999, Arch. Biochem. Biophys. 369:1-10. While some single cytochrome P450 monooxygenase enzymes can metabolize multiple substrates, many of these enzymes are highly substrate specific. The cytochrome P450 monooxygenase of the present invention refers to a P450 polypeptide useful in the production of 1-octen-3-ol in the presence of linoleic acid.

The term “1-octen-3-ol” refers to the 8 carbon volatile compound thought to be responsible, at least in part, for the “beany” and “grassy” soybean off-flavors and the desirable flavor of some cheeses and fresh milk as well as contributing to the characteristic flavor of mushrooms, green beans, tomatoes, cucumbers, and ripe fruit. 1-octen-3-ol is also known as mushroom alcohol, amyl vinyl carbinol, matsutake alcohol, oct-1-en-3-ol, 3-hydroxy-1-octene, and n-pentyl vinyl carbinol, among others. Until now a lipoxygenase and a hydroperoxide lyase were thought to be responsible for the production of (R)-1-octen-3-ol in mushrooms (Husson, F. et al., 2001, Process Biochem. 37:177-182). In the present application a soybean polynucleotide encoding a cytochrome P450 is shown to result in formation of 1-octen-3-ol from linoleic acid.

Production of 1-octen-3-ol may also be accomplished by transforming plant cells, insect cells, yeast cells, or other host cells with the polynucleotide of the invention. For example, as shown in Example 3 below, expression in yeast of a cytochrome P450 monooxygenase of the invention in the presence of linoleic acid produces 1-octen-3-ol. Expression of the polynucleotides of the invention in other host cells is well known to those familiar with the art. Vectors useful for expression in plant cells, insect cells, or yeast cells are known to those skilled in the art. For reviews on expression of polypeptides using insect cells and Baculoviruses see King, L. A., and Possee, R. D., 1992, The Baculovirus Expression System: A Laboratory Guide; New York, N.Y.: Chapman and Hall, eds.; O'Reilly, D. R., et al., 1992, Baculovirus Expression Vectors: A Laboratory Manual, New York, N.Y.: W. H. Freeman and Company, eds.; Richardson, C. D., 1995, Baculovirus Expression Protocols. In Methods in Molecular Biology, Vol. 39. (J. M. Walker, ed. Humana Press, Totowa, N.J.; among others.

Chemical oxidation of 1-octen-3-ol produces 1-octen-3-one. The levels of 1-octen-3-one increase in soybean white flake slurries as the levels of 1-octen-3-ol increase. Modulation of the levels of 1-octen-3-ol in soybean products should lead to the corresponding changes in 1-octen-3-one. Thus, the methods taught here to reduce 1-octen-3-ol levels in soybeans and soybean products may lead to concomitant reduction in the levels of 1-octen-3-one.

Polynucleotides of the instant invention may also be characterized by the percent identity of the amino acid sequences that they encode to the amino acid sequences disclosed herein, as determined by algorithms commonly employed by those skilled in this art. Suitable isolated polynucleotides of the present invention encode polypeptides that are at least about 70% identical, preferably at least about 80% identical to the amino acid sequences reported herein. Preferred nucleic acid fragments encode amino acid sequences that are at least about 85% identical to the amino acid sequences reported herein. More preferred nucleic acid fragments encode amino acid sequences that are at least about 90% identical to the amino acid sequences reported herein. Most preferred are nucleic acid fragments that encode amino acid sequences that are at least about 95% identical to the amino acid sequences reported herein. Sequence alignments and percent identity calculations were performed using the Megalign program of the LASERGENE bioinformatics computing suite (DNASTAR Inc., Madison, Wis.). Multiple alignment of the sequences was performed using the Clustal V method of alignment (Higgins, D. G. and Sharp, P. M., 1989, Comput. Appl. Biosci. 5:151-153; Higgins, D. G. et al., 1992, Comput. Appl. Biosci. 8:189-191) using the default parameters (GAP PENALTY=10, GAP LENGTH PENALTY=10). Default parameters for pairwise alignments using the Clustal V method were KTUPLE 1, GAP PENALTY=3, WINDOW=5 and DIAGONALS SAVED=5.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequence encoding the cytochrome P450s of the invention as set forth in SEQ ID NO:3. In addition, the present invention concerns an isolated polynucleotides comprising SEQ ID NO: 2, or a portion thereof capable of suppressing production of 1-octen-3-ol when introduced into a cell that normally produces 1-octen-3-ol. The skilled artisan is well aware of the “codon-bias” exhibited by a specific host cell in usage of nucleotide codons to specify a given amino acid. Therefore, when synthesizing a polynucleotide for improved expression of a specific gene in a host cell, it is desirable to design the polynucleotide such that its frequency of codon usage approaches the frequency of preferred codon usage of the host cell.

The terms “homology”, “homologous”, “substantially similar” and “corresponding substantially” are used interchangeably herein. They refer to nucleic acid fragments wherein changes in one or more nucleotide bases does not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is well understood by those skilled in the art that heterogeneity, represented mainly in single nucleotide polymorphisms, is common for different soybean cultivars (Jack and Wye, for example) It is therefore understood, as those skilled in the art will appreciate, that the invention encompasses more than the specific exemplary sequences.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized,” as related to nucleic acid fragment, means that the component nucleotides were assembled in vitro. Manual chemical synthesis of nucleic acid fragments may be accomplished using well-established procedures, or automated chemical synthesis can be performed using one of a number of commercially available machines. Accordingly, the nucleic acid fragments can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that is capable of directing expression of a specific protein or functional RNA.

An “allele” is one of several alternative forms of a gene occupying a given locus on a chromosome. When the alleles present at a given locus on a pair of homologous chromosomes in a diploid plant are the same that plant is “homozygous” at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

The term “endogenous gene” refers to a native gene in its natural location in the genome of an organism. The terms “foreign gene” and “transgene” are used interchangeably herein and refer to a gene not normally found in the host organism that is introduced into the host organism by transformation or gene transfer. Transgenes may become physically inserted into a genome of the host cell (i.e., through recombination) or may be maintained outside of a genome of the host cell (i.e., on an extrachromosomal element).

The choice of recombinant expression construct is dependent upon the method that will be used to transform host cells. The skilled artisan is well aware of the genetic elements that must be present on the recombinant expression construct in order to successfully transform, select and propagate host cells. The skilled artisan will also recognize that different independent transformation events may be screened to obtain lines displaying the desired expression level and pattern. Such screening may be accomplished by, but is not limited to, Southern analysis of DNA, Northern analysis of mRNA expression, Western analysis of protein expression, or phenotypic analysis.

The term “coding sequence” refers to a DNA fragment that codes for a structural RNA, or for a polypeptide having a specific amino acid sequence. The boundaries of a protein coding sequence are generally determined by a ribosome binding site (prokaryotes) or by a start codon (eukaryotes) located at the 5′ end of the mRNA and a transcription terminator sequence located just downstream of the open reading frame at the 3′ end of the mRNA. A coding sequence can include, but is not limited to, DNA, cDNA, exons, and recombinant nucleic acid sequences. “Regulatory sequences” refer to nucleotides located upstream (5′ non-coding sequences), within, or downstream (3′ non-coding sequences) of a coding sequence, which may influence the transcription, RNA processing, stability, or translation of the associated coding sequence. Regulatory sequences may include, but are not limited to, promoters, translation leader sequences, introns, and polyadenylation recognition sequences.

“Promoter” refers to a region of DNA capable of controlling the expression of a coding sequence or functional RNA. The promoter may consist of proximal and more distal upstream elements. These upstream elements include, but are not limited to, enhancers, repressor binding motifs, tissue-specific motifs, developmental responsive motifs, and hormone responsive motifs. An “enhancer” is a region of DNA capable of stimulating promoter activity. These upstream elements may be innate regions of the promoter or a heterologous element inserted to enhance the level or tissue-specificity of a promoter.

A number of promoters can be used in the practice of the present invention. The promoters can be selected based on the desired outcome. Nucleic acids used to accomplish the invention can be combined in any host organism with a promoter or element that has constitutive, tissue-specific, inducible, or other gene regulatory activities.

In some embodiments, promoters or enhancers can be used or modified to accomplish the present invention. For example, endogenous promoters can be altered in vivo by mutation, deletion, and/or substitution (see for example U.S. Pat. No. 5,565,350. Gene expression can be modulated under conditions suitable for host cell growth so as to alter the total concentration and/or alter the composition of the polypeptides of the present invention in host cell.

“Tissue-specific” promoters direct RNA production preferentially in particular types of cells or tissues. Promoters that cause a gene to be expressed in most cell types at most times are commonly referred to as “constitutive promoters.” New promoters of various types useful in plant cells are constantly being discovered; the compilation by Okamuro, J. K. and Goldberg, R. B. (1989, Biochemistry of Plants 15:1-82) provides numerous examples. It is further recognized that since in most cases the exact boundaries of regulatory sequences have not been completely defined, DNA fragments of some variation may have identical promoter activity.

The “translation leader sequence” or “leader” refers to a polynucleotide sequence located upstream or 5′ of the coding sequence. The translation leader sequence is present in the fully processed mRNA upstream of the translation start site. The translation leader sequence may affect processing of the primary transcript to mRNA, mRNA stability or translation efficiency. Examples of translation leader sequences have been described (Turner, R. and Foster, G. D., 1995, Mol. Biotechnol. 3:225-236).

Commonly used promoters include, but are not limited to, the nopaline synthase (NOS) promoter (Ebert et al., 1987, Proc. Natl. Acad. Sci. U.S.A. 84:5745-5749), the octapine synthase (OCS) promoter, caulimovirus promoters such as the cauliflower mosaic virus (CaMV) 19S promoter (Lawton et al., 1987, Plant Mol. Biol. 9:315-324), the CaMV 35S promoter (Odell et al., 1985, Nature 313:810-812), and the figwort mosaic virus 35S promoter, the light inducible promoter from the small subunit of rubisco, the Adh promoter (Walker et al., 1987, Proc. Natl. Acad. Sci. U.S.A. 84:6624-66280), the sucrose synthase promoter (Yang et al., 1990, Proc. Natl. Acad. Sci. U.S.A. 87:4144-4148), the R gene complex promoter (Chandler et al., 1989, Plant Cell 1:1175-1183), and the chlorophyll a/b binding protein gene promoter. Other commonly used promoters are, the promoters for the potato tuber ADPGPP genes, the granule bound starch synthase promoter, the glutelin gene promoter, the maize waxy promoter, Brittle gene promoter, the Shrunken 2 promoter, the acid chitinase gene promoter, and the zein gene promoters (15 kD, 16 kD, 19 kD, 22 kD, and 27 kD; Perdersen et al., 1982, Cell 29:1015-1026). A plethora of promoters is described in PCT Publication No. WO 00/18963, published on Apr. 6, 2000.

The “3′ non-coding region” or “terminator region” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989, Plant Cell 1:671-680).

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into protein by the cell. “cDNA” refers to a DNA that is complementary to and derived from an mRNA. The cDNA can be single-stranded or converted into the double stranded form using, for example, the Klenow fragment of DNA polymerase I. “Sense” RNA refers to RNA transcript that includes the mRNA and so can be translated into a polypeptide by the cell. “Antisense RNA” refers to an RNA transcript that is complementary to all or part of a target primary transcript or mRNA and that blocks the expression of a target gene (see U.S. Pat. No. 5,107,065). The complement of an antisense RNA may be with any part of the specific gene transcript, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The term “operably linked” and “under the control of” refer to the association of nucleic acid fragments on a single polynucleotide so that the function of one is affected by the function of the other. For example, a promoter is operably linked with a coding sequence when it is capable of affecting the expression of that coding sequence (i.e., that the coding sequence is under the transcriptional control of the promoter). Similarly, a polynucleotide may be under the control of a promoter that is capable of affecting the expression of the polynucleotide. Coding sequences can be operably linked to regulatory sequences in sense or antisense orientation.

The terms “recombinant construct,” “expression construct,” “chimeric construct,” “construct,” “recombinant DNA construct,” and “recombinant DNA fragment” are used interchangeably herein and are nucleic acid fragments. A recombinant construct comprises an artificial combination of nucleic acid fragments, including, and not limited to, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. Screening to obtain lines displaying the desired expression level and pattern may be accomplished by Southern analysis of DNA, Northern analysis of mRNA expression, RT-PCR, immunoblotting analysis of protein expression, or phenotypic analysis, among others.

As used herein, the term “cassette” refers to a recombinant DNA construct comprising a promoter operably linked to a nucleic acid sequence of interest, which is followed by a termination signal and which is surrounded by at least one restriction endonuclease site so that it may be removed from other fragments in the same construct.

The term “recombinant DNA construct” refers to a DNA construct assembled from nucleic acid fragments obtained from different sources. The types and origins of the nucleic acid fragments may-be very diverse. A “recombinant DNA construct” includes and is not limited to the following combinations: a) nucleic acid fragment corresponding to a promoter operably linked to at least one nucleic acid fragment encoding a selectable marker, followed by a nucleic acid fragment corresponding to a terminator, b) a nucleic acid fragment corresponding to a promoter operably linked to a nucleic acid fragment capable of producing a stem-loop structure, and followed by a nucleic acid fragment corresponding to a terminator, and c) any combination of a) and b) above. In the stem-loop structure at least one nucleic acid fragment that is capable of suppressing expression of a native gene comprises the “loop” and is surrounded by nucleic acid fragments capable of producing a stem.

The term “expression,” as used herein refers to the transcription and stable accumulation of sense (mRNA) or antisense RNA derived from a polynucleotide of the invention. Expression may also refer to translation of mRNA into a polypeptide. “Suppression” refers to gene product production in transgenic organisms that is reduced compared to levels of production in wild-type organisms. “Antisense inhibition” refers to the production of antisense RNA transcripts capable of suppressing the expression of the target protein. “Overexpression” refers to the production of a gene product in transgenic organisms that exceeds levels of production in wild-type organisms. “Co-suppression” refers to the production of sense RNA transcripts capable of suppressing the expression of identical or substantially similar foreign or endogenous genes (U.S. Pat. No. 5,231,020). One can also envision the use of “RNAi” related techniques to reduce the expression of the genes of the present invention. See for example U.S. Pat. No. 6,506,559. Such techniques rely on the use of constructs resulting in the accumulation of double stranded RNA with one strand complementary to the target gene to be silenced.

“Altered levels” of a polypeptide, protein, or enzyme refers to amounts of the polypeptide, protein, or enzyme or the activity of such polypeptide, protein, or enzyme that differ from those of wild-type organisms. “Wild-type” organisms also are referred to herein as “non-transformed” and “non-mutated.” “Wild-type” organisms may have been transformed with another transgene or have a mutation in another gene, but do not have a mutation in or have not been transformed with the cytochrome P450 monooxygenase of the invention. The polynucleotides of the present invention may be used to prepare recombinant DNA constructs that may be introduced into host cells to create transgenic cells having altered levels or activity of the novel cytochrome P450 monooxygenase.

In the present invention “altered levels” may also refer to amounts of 1-octen-3-ol that differ from amounts found in wild-type organisms. Altered levels of a polypeptide, protein, enzyme, or 1-octen-3-ol include an increase and a decrease in gene product amounts compared to wild-type organisms. Accordingly, altered includes increase, enhance, amplify, multiply, elevate, raise, and the like as well as decrease, reduce, lower, prevent, inhibit, stop, eliminate, and the like.

“Mature” protein refers to a post-translationally processed polypeptide; i.e., one from which any pre- or pro-peptides present in the primary translation product has been removed. “Precursor” protein refers to the primary product of translation of mRNA; i.e., with pre- and pro-peptides still present. Pre- and pro-peptides may be but are not limited to intracellular localization signals.

A “signal peptide” is an amino acid sequence that is translated in conjunction with a protein and directs the protein to the secretory system (Chrispeels, M., 1991, Ann. Rev. Plant Phys. Plant Mol. Biol. 42:21-53).

“Transformation” refers to the transfer of a nucleic acid fragment into the genome of a host organism, resulting in genetically stable inheritance. Host organisms containing the transformed nucleic acid fragments are referred to as “transgenic” organisms. “Host cell” refers the cell into which transformation of the recombinant DNA construct takes place and may include a yeast cell, a bacterial cell, an insect cell and a plant cell. Examples of methods of plant transformation include Agrobacterium-mediated transformation (De Blaere et al., 1987, Meth. Enzymol. 143:277) and particle-accelerated or “gene gun” transformation technology (Klein et al., 1987, Nature (London) 327:70-73; U.S. Pat. No. 4,945,050), among others.

Expression of a cytochrome P450 of the invention, for example, results in production of a level of the encoded cytochrome P450 protein in a transformed host cell that is altered as compared to the level produced in a non-transformed host cell. Also, a transgenic plant, or plant part, comprising a polynucleotide of the present invention, such as for example, at least a portion of SEQ ID NO:2, under the control of a heterologous promoter results in plants having altered levels of 1-octen-3-ol. Plants may be selected from the group consisting of monocots and dicots. Monocots include and are not limited to corn, oat, rice, wheat, barley, palm, and the like. Dicots include and are not limited to Arabidopsis, soybean, oilseed Brassica, peanut, sunflower, safflower, cotton, tobacco, tomato, potato, cocoa, and the like. Plant parts include and are not limited to seeds, grains, leaves, and roots, for example.

Isolated polynucleotides of the present invention can be incorporated into recombinant DNA constructs capable of introduction into and replication in a host cell. A “vector” may be such a construct that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Flevin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989 (hereinafter “Sambrook”).

“PCR” or “polymerase chain reaction” is a technique for the synthesis of large quantities of specific DNA segments. It consists of a series of repetitive cycles (Perkin Elmer Cetus Instruments, Norwark, Conn.). Typically, the double-stranded DNA is heat denatured, the two primers complementary to the 3′ boundaries of the target segments are annealed at low temperature and then extended at an intermediate temperature. One set of these three consecutive steps is referred to asacycle.

Amino acid and nucleotide sequences may be evaluated manually by one skilled in the art, or by using computer-based sequence comparison and identification tools that employ algorithms such as BLAST (Basic Local Alignment Search Tool; Altschul et al. (1993) J. Mol. Biol. 215:403-410; see also the explanation of the BLAST algorithm on the world wide web site for the National Center for Biotechnology Information at the National Library of Medicine of the National Institutes of Health). In general, a sequence of ten or more contiguous amino acids or thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising 30 or more contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12 or more nucleotides may be used as amplification primers in PCR in order to obtain a particular nucleic acid fragment comprising the primers. The instant specification teaches amino acid and nucleotide sequences encoding P450 monooxygenases. The skilled artisan, having the benefit of the sequences as reported herein, may now use all or a portion of the disclosed sequences for purposes known to those skilled in this art. Accordingly, the instant invention comprises the complete sequences as reported in the accompanying Sequence Listing, as well as portions of those sequences that may be used in suppressing the P450s of the invention using protocols well known to those of skill in the art.

Genes encoding other cytochrome P450s similar to the Euphorbia lagascae CYP726A1, either as cDNAs or genomic DNAs, may be isolated directly by using all or a portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Sambrook). Moreover, the entire sequences can be used directly to synthesize DNA probes by methods known to the skilled artisan such as random primer DNA labeling, nick translation, or end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full-length cDNA or genomic fragments under conditions of appropriate stringency.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman, M. A. et al., 1988, Proc. Natl. Acad. Sci. U.S.A. 85:8998-9002) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., 1989, Proc. Natl. Acad. Sci. U.S.A 86:5673-5677; Loh et al., 1989, Science 243:217-220). Products generated by the 3′ and 5′ RACE procedures can be combined to generate full-length cDNAs (Frohman, M. A. and Martin, G. R., 1989, Techniques 1:165).

Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner, 1984, Adv. Immunol. 36:1-34; Sambrook).

The polynucleotides of the instant invention may be used to create transgenic plants in which the cytochrome P450 of the present invention is present at lower or higher levels than normal or in cell types or developmental stages in which they are not normally found. This would have the effect of altering production of 1-octen-3-ol in those cells. It is believed that suppression of the polynucleotides of the invention in soybeans will result in more palatable soybeans and soybean products. Overexpression of the cytochrome P450s of the invention in mushrooms may lead to the production of more 1-octen-3-ol. In the flavor industry it may be possible to use the cytochrome P450s of the invention to produce 1-octen-3-ol that may be added to foods, such as soups and cheeses, to enhance their flavor. Sensory studies of the R-(−)-1-octen-3-ol and (S)-(+)-1-octen-3-ol enantiomers showed that the mushroom-like odor was exclusively caused by the R-(−)-enantiomer (Mosandl, G. H. and Gessner, M, 1986, J. Agric. Food Chem. 34; 119-122). Thus an advantage to biologically synthesized 1-octen-3-ol as compared to the current chemically synthesized 1-octen-3-ol is that the biologically synthesized material would be enantiomerically pure which may be expected to improve the quality of the flavoring. Overexpression of the cytochrome P450 proteins of the instant invention may be accomplished by first constructing a recombinant DNA construct in which the coding region is operably linked to a promoter capable of directing its expression to the desired tissues at the desired stage of development. The recombinant DNA construct may comprise promoter sequences and translation leader sequences derived from the same genes. 3′ non-coding sequences encoding transcription termination signals may also be provided. The instant recombinant DNA construct may also comprise one or more introns in order to facilitate gene expression. Examples of possible recombinant DNA constructs useful in overexpression or suppression of the instant cytochrome P450s are shown in Example 5, below. The term “overexpression” includes increase, augment, amplify, raise, enhance, and the like.

Plasmid vectors comprising the isolated polynucleotide of the invention may be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene. Screening to identify transformed host cells containing the desired gene may be accomplished by Southern analysis of DNA, Northern analysis of mRNA expression, RT-PCR of RNA, Western analysis of protein expression, or phenotypic analysis.

For some applications it may be useful to direct the instant polypeptides to different cellular compartments, or to facilitate their secretion from the cell. It is thus envisioned that the recombinant DNA constructs described above may be further supplemented by altering the coding sequence to encode appropriate intracellular targeting signals such as transit signals (Keegstra, 1989, Cell 56:247-253), signal sequences with or without endoplasmic reticulum retention signals (Chrispeels, 1991, Ann. Rev. Plant Phys. Plant Mol. Biol. 42:21-53), or nuclear localization signals (Raikhel, N., 1992, Plant Phys. 100:1627-1632) with or without removing targeting signals that are already present. While the references cited give examples of each of these, the list is not exhaustive and more targeting signals of utility may be discovered in the future.

As stated herein, “suppression” refers to the reduction of the level of enzyme activity detectable in a transgenic plant when compared to the level of enzyme activity detectable in a plant with the native enzyme. The level of enzyme activity in a plant with the native enzyme is referred to herein as “wild type” activity. The term “suppression” includes lower, reduce, decline, decrease, inhibit, eliminate and prevent. This reduction may be due to the decrease in translation of the native mRNA into an active enzyme. It may also be due to the transcription of the native DNA into decreased amounts of mRNA and/or to rapid degradation of the native mRNA. The term “native enzyme” refers to an enzyme that is produced naturally in the desired cell.

Another aspect of the present invention is directed to a method of suppressing activity of a native cytochrome P450 monooxygenase in soybean to obtain soybeans producing reduced levels of 1-octen-3-ol.

Suppression of the cytochrome P450s of the invention in plants may be accomplished by any one of many methods known in the art which include the following. “Cosuppression” refers to the production of sense RNA transcripts capable of suppressing the expression of identical or substantially similar native genes (U.S. Pat. No. 5,231,020). Co-suppression constructs in plants have been previously designed by focusing on overexpression of a nucleic acid sequence having homology to a native mRNA, in the sense orientation, which results in the reduction of all RNA having homology to the overexpressed sequence (see Vaucheret et al. (1998) Plant J. 16:651-659; and Gura (2000) Nature 404:804-808). “Antisense inhibition” refers to the production of antisense RNA transcripts capable of suppressing the expression of the target protein. Plant viral sequences may be used to direct the suppression of proximal mRNA encoding sequences (PCT Publication WO 98/36083 published on Aug. 20, 1998). “Hairpin” structures that incorporate all, or part, of an mRNA encoding sequence in a complementary orientation resulting in a potential “stem-loop” structure for the expressed RNA have been described (PCT Publication WO 99/53050 published on Oct. 21, 1999). In this case the stem is formed by polynucleotides corresponding to the gene of interest inserted in either sense or anti-sense orientation with respect to the promoter and the loop is formed by some polynucleotides of the gene of interest, which do not have a complement in the construct. This increases the frequency of cosuppression or silencing in the recovered transgenic plants. For review of hairpin suppression see Wesley, S. V. et al. (2003) Methods in Molecular Biology, Plant Functional Genomics: Methods and Protocols 236:273-286. A construct where the stem is formed by at least 30 nucleotides from a gene to be suppressed and the loop is formed by a random nucleotide sequence has also effectively been used for suppression (WO 99/61632 published on Dec. 2, 1999). The use of poly-T and poly-A sequences to generate the stem in the stem-loop structure has also been described (WO 02/00894 published Jan. 3, 2002). Yet another variation includes using synthetic repeats to promote formation of a stem in the stem-loop structure. Transgenic organisms prepared with such recombinant DNA fragment show reduced levels of the protein encoded by the polynucleotide from which the nucleotide fragment forming the loop is derived as described in PCT Publication WO 02/00904, published Jan. 3, 2002. The use of constructs that result in dsRNA has also been described. In these constructs convergent promoters direct transcription of gene-specific sense and antisense RNAs inducing gene suppression (see for example Shi, H. et al. (2000) RNA 6:1069-1076; Bastin, P. et al. (2000) J. Cell Sci. 113:3321-3328; Giordano, E. et al. (2002) Genetics 160:637-648; LaCount, D. J. and Donelson, J. E. US patent Application No. 20020182223, published Dec. 5, 2002; Tran, N. et al. (2003) BMC Biotechnol. 3:21; and Applicant's U.S. Provisional Application No. 60/578,404, filed Jun. 9, 2004).

Other methods for suppressing an enzyme include, but are not limited to, use of polynucleotides that may form a catalytic RNA or may have ribozyme activity (U.S. Pat. No. 4,987,071 issued Jan. 22, 1991), and micro RNA (also called miRNA) interference (Javier et al. (2003) Nature 425:257-263).

A variety of nucleic acid amplification-based methods of genetic and physical mapping may be carried out using the instant nucleic acid sequences. Examples include, and are not limited to, allele-specific amplification (Kazazian, H. H. Jr, 1989, J. Lab. Clin. Med. 11:95-96), polymorphism of PCR-amplified fragments (CAPS; Sheffield, V. C., et al., 1993, Genomics 16:325-332), allele-specific ligation (Landegren, U., et al., 1988, Science 241:1077-1080), nucleotide extension reactions (Sokolov, B. P., 1990, Nucleic Acid Res. 18:3671), radiation hybrid mapping (Walter, M. A. et al., 1994, Nat. Genet. 7:22-28), fluorescence in situ hybridization (FISH; Svitashev, S. K. and Somers, D. A., 2002, Plant Cell Tissue Organ Cult. 69:205-214), and Happy Mapping (Dear, P. H. and Cook, P. R., 1989, Nucleic Acid Res. 17:6795-6807). For these methods, the sequence of a nucleic acid fragment is used to design and produce primer pairs for use in the amplification reaction or in primer extension reactions. Design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for all mapping methods.

A “portion capable of increasing the level of 1-octen-3-ol” and a “portion capable of decreasing the level of 1-octen-3-ol” refers to a portion or subfragment of an isolated nucleic acid fragment in which the ability to alter gene expression or produce a certain phenotype is retained whether or not the fragment or subfragment may be translated into an active enzyme.

For example, a portion may be a portion capable of suppressing expression of a native gene. A fragment or subfragment may be used in the design of chimeric genes or recombinant DNA constructs to produce the desired phenotype in a transformed plant. Chimeric genes may be designed for use in suppression by linking a nucleic acid fragment or subfragment thereof, whether or not it is translated into an active enzyme, in the sense or antisense orientation relative to a plant promoter sequence. Recombinant DNA fragments may be designed to comprise nucleic acid fragments capable of promoting formation of a stem-loop structure. In a stem-loop structure either the loop or the stem comprises a portion of the gene to be suppressed. The nucleic acid fragment should have a stretch of at least about 20 contiguous nucleotides that are identical to the gene to be suppressed. The stretch of contiguous nucleotides may be any number, from at least about 20, or about 22, or about 25, or about 32, to the size of the entire gene to be suppressed.

Methods for transforming dicots and obtaining transgenic plants have been published, among others, for cotton (U.S. Pat. No. 5,004,863, U.S. Pat. No. 5,159,135); soybean (U.S. Pat. No. 5,569,834, U.S. Pat. No. 5,416,011); Brassica (U.S. Pat. No. 5,463,174); peanut (Cheng et al., 1996, Plant Cell Rep. 15:653-657, McKently et al., 1995, Plant Cell Rep. 14:699-703); papaya (Ling, K. et al., 1991, Bio/technology 9:752-758); and pea (Grant et al., 1995, Plant Cell Rep. 15:254-258). For a review of other commonly used methods of plant transformation see Newell, C. A., 2000, Mol. Biotechnol. 16:53-65. One of these methods of transformation uses Agrobacterium rhizogenes (Tepfler, M. and Casse-Delbart, F., 1987, Microbiol. Sci. 4:24-28). Transformation of soybeans using direct delivery of DNA has been published using PEG fusion (PCT publication WO 92/17598), electroporation (Chowrira, G. M. et al., 1995, Mol. Biotechnol. 3:17-23; Christou, P. et al., 1987, Proc. Natl. Acad. Sci. U.S.A. 84:3962-3966), microinjection, or particle bombardment (McCabe, D. E. et. al., 1988, BiolTechnology 6:923; Christou et al., 1988, Plant Physiol. 87:671-674).

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, 1988, In.: Methods for Plant Molecular Biology, (Eds.), Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. The regenerated plants may be self-pollinated. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide(s) is cultivated using methods well known to one skilled in the art.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook; Maliga et al., 1995, Methods in Plant Molecular Biology, Cold Spring Harbor Press; Birren et al., 1998, Genome Analysis: Detecting Genes, 1, Cold Spring Harbor, New York; Birren et al., 1998, Genome Analysis: Analyzing DNA, 2, Cold Spring Harbor, New York; Plant Molecular Biology: A Laboratory Manual, eds. Clark, Springer, New York,1997).

The terms “soy” and “soybean” are used interchangeably herein. Within the scope of the invention are soybean plants (Glycine soja or Glycine max), seeds, and plant parts obtained from such transformed plants. Also within the scope of the invention are soybean products derived from the transformed plants such as grain, protein products, oils, and products including such soybean products like feed and foodstuffs. Plant parts include differentiated and undifferentiated tissues, including and not limited to, roots, stems, shoots, leaves, pollen, seeds, tumor tissue, and various forms of cells and cultures such as single cells, protoplasts, embryos, and callus tissue. The plant tissue may be in plant or in organ, tissue or cell culture.

While not intending to be bound by any theory or theories of operation, it is believed by those of skill in the art that altered levels of 1-octen-3-ol in foods have different effects. Foods originating from soybean plants having a decreased level of 1-octen-3-ol are believed to be better-tasting foods. Foods having an increased level of 1-octen-3-ol, such as, and not limited to soups, cheeses, and mushroom-containing foods, are also believed to be better-tasting. Accordingly, plants grown with altered levels of the cytochrome P450s of the invention may contribute to better-flavored foods. Thus, also included in the invention are the grains from the transgenic plants of the invention.

Included within the scope of this invention are soybean products that include protein isolates, protein concentrates, food products, feed products, etc. Methods for obtaining such products are well-known to those skilled in the art. For example soybean protein products can be obtained in a variety of ways. Conditions typically used to prepare soy protein isolates have been described by (Cho, et al., 1981, U.S. Pat. No. 4,278,597; Goodnight, et al., 1978, U.S. Pat. No. 4,072,670). Soy protein concentrates are produced by three basic processes: acid leaching (at about pH 4.5), extraction with alcohol (about 55-80%), and denaturing the protein with moist heat prior to extraction with water. Conditions typically used to prepare soy protein concentrates have been described by Pass (1975, U.S. Pat. No. 3,897,574) and Campbell et al. (1985, in New Protein Foods, ed. by Altschul and Wilcke, Academic Press, Vol. 5, Chapter 10, Seed Storage Proteins, pp 302-338).

“Soybean-containing products” can be defined as those items produced of seeds from a suitable plant which are used in feeds, foods and/or beverages. For example, “soy protein products” can include, and are not limited to, those items listed in Table 1. “Soy protein products”.

TABLE 1
Soy Protein Products Derived from Soybean Seeds^{a}

Whole Soybean Products
Roasted Soybeans
Baked Soybeans
Soy Sprouts
Soy Milk
Specialty Soy Foods/Ingredients
Soy Milk
Tofu
Tempeh
Miso
Soy Sauce
Hydrolyzed Vegetable Protein
Whipping Protein
Processed Soy Protein Products
Full Fat and Defatted Flours
Soy Grits
Soy Hypocotyls
Soybean Meal
Soy Milk
Soy Protein Isolates
Soy Protein Concentrates
Textured Soy Proteins
Textured Flours and Concentrates
Textured Concentrates
Textured Isolates
^{a}See Soy Protein Products: Characteristics, Nutritional Aspects and Utilization (1987). Soy Protein Council.

“Processing” refers to any physical and chemical methods used to obtain the products listed in Table 1 and includes, and is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992). Products referred to as being “high protein” or “low protein” are those as described by these Standard Specifications. “NSI” refers to the Nitrogen Solubility Index as defined by the American Oil Chemists' Society Method Ac4 41. “KOH Nitrogen Solubility” is an indicator of soybean meal quality and refers to the amount of nitrogen soluble in 0.036 M KOH under the conditions as described by Araba and Dale (1990, Poult. Sci. 69:76-83). “White” flakes refer to flaked, dehulled cotyledons that have been defatted and treated with controlled moist heat to have an NSI of about 85 to 90. This term can also refer to a flour with a similar NSI that has been ground to pass through a No. 100 U.S. Standard Screen size. “Cooked” refers to a soy protein product, typically a flour, with an NSI of about 20 to 60. “Toasted” refers to a soy protein product, typically a flour, with an NSI below 20. “Grits” refer to defatted, dehulled cotyledons having a U.S. Standard screen size of between No. 10 and 80. “Soy Protein Concentrates” refer to those products produced from dehulled, defatted soybeans by three basic processes: acid leaching (at about pH 4.5), extraction with alcohol (about 55-80%), and denaturing the protein with moist heat prior to extraction with water. Conditions typically used to prepare soy protein concentrates have been described by Pass (1975, U.S. Pat. No. 3,897,574; Campbell et al., 1985, in New Protein Foods, ed. by Altschul and Wilcke, Academic Press, Vol. 5, Chapter 10, Seed Storage Proteins, pp 302-338). “Extrusion” refers to processes whereby material (grits, flour or concentrate) is passed through a jacketed auger using high pressures and temperatures as a means of altering the texture of the material. “Texturing” and “structuring” refer to extrusion processes used to modify the physical characteristics of the material. The characteristics of these processes, including thermoplastic extrusion, have been described previously (Atkinson, 1970, U.S. Pat. No. 3,488,770, Horan, 1985, In New Protein Foods, ed. by Altschul and Wilcke, Academic Press, Vol. 1A, Chapter 8, pp 367-414). Moreover, conditions used during extrusion processing of complex foodstuff mixtures that include soy protein products have been described previously (Rokey, 1983, Feed Manufacturing Technology III, 222-237; McCulloch, U.S. Pat. No. 4,454,804).

Also, within the scope of this invention are food, food supplements, food bars, and beverages that have incorporated therein a soybean-derived product of the invention. The beverage can be in a liquid or in a dry powdered form.

The foods to which the soybean-derived product of the invention can be incorporated/added include almost all foods/beverages. For example, there can be mentioned meats such as ground meats, emulsified meats, marinated meats, and meats injected with a soybean-derived product of the invention. Included may be beverages such as nutritional beverages, sports beverages, protein-fortified beverages, juices, milk, milk alternatives, and weight loss beverages. Mentioned may also be cheeses such as hard and soft cheeses, cream cheese, and cottage cheese. Included may also be frozen desserts such as ice cream, ice milk, low fat frozen desserts, and non-dairy frozen desserts. Finally, yogurts, soups, puddings, bakery products, salad dressings, spreads, and dips (such as mayonnaise and chip dips) may be included. The soybean-derived product can be added in an amount selected to deliver a desired dose to the consumer of the food and/or beverage.

In still another aspect this invention concerns a method of producing an soybean-derived product which comprises: (a) cracking the seeds obtained from transformed plants of the invention to remove the meats from the hulls; and (b) flaking the meats obtained in step (a) to obtain the desired flake thickness.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated by reference in its entirety.

### EXAMPLES

### Example 1

### Preparation of cDNA Libraries and Sequencing of cDNA Inserts

cDNA libraries representing mRNAs from various soybean tissues were prepared in Uni-ZAP™ XR vectors according to the manufacturer's protocol (Stratagene Cloning Systems, La Jolla, Calif.). Conversion of the Uni-ZAP™ XR libraries into plasmid libraries was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences or plasmid DNA was prepared from cultured bacterial cells. Amplified insert DNAs or plasmid DNAs were sequenced in dye-primer sequencing reactions to generate partial cDNA sequences (expressed sequence tags or “ESTs”; see Adams, M. D. et al., 1991, Science 252:1651). The resulting ESTs were analyzed using a Perkin Elmer Model 377 fluorescent sequencer.

Full-insert sequence (FIS) data was generated utilizing a modified transposition protocol. Clones identified for FIS were recovered from archived glycerol stocks as single colonies, and plasmid DNAs were isolated via alkaline lysis. Isolated DNA templates were reacted with vector primed M13 forward and reverse oligonucleotides in a PCR-based sequencing reaction and loaded onto automated sequencers. Confirmation of clone identification was performed by sequence alignment to the original EST sequence from which the FIS request was made.

Confirmed templates were transposed via the Primer Island transposition kit (PE Applied Biosystems, Foster City, Calif.) which is based upon the Saccharomyces cerevisiae Ty1 transposable element (Devine, S. E. and Boeke, J. D., 1994, Nucleic Acids Res. 22:3765-3772). The in vitro transposition system places unique binding sites randomly throughout a population of large DNA molecules. The transposed DNA was then used to transform DH10B electro-competent cells (Gibco BRL/Life Technologies, Rockville, Md.) via electroporation. The transposable element contains an additional selectable marker (named DHFR; Fling, M. E. and Richards, C., 1983, Nucleic Acids Res. 11:5147-5158), allowing for dual selection on agar plates of only those clones containing the integrated transposon. Multiple clones were randomly selected from each transposition reaction, plasmid DNAs were prepared via alkaline lysis, and templates were sequenced using ABI Prism dye-terminator ReadyReaction mix (Applied Biosystems, Foster City, Calif.), outward from the transposition event site, utilizing unique primers specific to the binding sites within the transposon.

Sequence data was collected (ABI Prism Collections, Applied Biosystems, Foster City, Calif.) and assembled using Phred and Phrap (Ewing, B., et al., 1998, Genome Res. 8:175-185; Ewing, B. and Green, P., 1998, Genome Res. 8:186-194). Phred re-reads the ABI sequence data, re-calls the bases, assigns quality values, and writes the base calls and quality values into editable output files. The Phrap sequence assembly program uses the quality values assigned by Phred to increase the accuracy of the assembled sequence contigs. Assemblies were viewed using the Consed sequence editor (Gordon, D., et al., 1998, Genome Res. 8:195-202).

### Example 2

### Identification of cDNA Clones Encoding Polypeptides Similar to Euphorbia lagascae CYP726A1

While it is known that the compound 1-octen-3-ol accumulates in soybean seeds, the biosynthetic pathway is not known. However, it seems likely that linoleic acid is the precursor. A cytochrome P450 monooxygenase (CYP726A1) that catalyzes the conversion of linoleic acid to vernolic acid was characterized from Euphorbia lagascae (Cahoon, E. B., et al., 2002, Plant Phys. 128:615-624). We hypothesized that a similar enzyme may be involved in the conversion of linoleic acid into 1-octen-3-ol.

A search for nucleic acids from Glycine max (soybean) that encode polypeptides with similarity to the amino acid sequence of the Euphorbia lagascae CYP726A1 (SEQ ID NO:1; NCBI Accession No. ML62063.1) was carried out using a tBLASTn search against a proprietary database containing contigs assembled from ESTs and/or full-insert sequences of soybean cDNAs from both public and private sources. Contigs are nucleotide sequences assembled from constituent nucleotide sequences that share common or overlapping regions of sequence identity. The tBLASTn algorithm is used to search an amino acid query against a nucleotide database that is translated in all 6 reading frames.

This tBLASTn analysis resulted in several contigs encoding polypeptides with significant homology to the Euphorbia lagascae CYP726A1. The nucleotide sequence of the entire cDNA insert in clone sfl1.pk0045.g7 is part of one such contig, and the polynucleotide sequence of sfl1.pk0045.g7 encompasses the complete contig. Also included in this contig is a soybean EST having NCBI accession Number gm700018b10d7. This EST is identical to nucleotides 1034 through 1697 of the sfl1.pk0045.g7 full insert sequence.

In Example 3 below, the sfl1.pk0045.g7 full insert sequence polynucleotide is shown to produce 1-octen-3-ol when expressed in yeast in the presence of linoleic acid.

Clone sfl1.pk0045.g7 is derived from a library prepared from soybean (Glycine max L., Wye) immature flowers. The sequence of the entire cDNA insert in clone sfl1.pk0045.g7 is shown in SEQ ID NO:2. The deduced amino acid sequence obtained from translating nucleotides 22 through 1548 of SEQ ID NO:2 is shown in SEQ ID NO:3.

The amino acid sequence shown in SEQ ID NO:3 was used in a BLASTP analysis against the NCBI “nr” database that is populated with non-redundant coding sequence translations found in GenBank, PDB, SwissProt, PIR, and PRF. This BLAST analysis showed that the amino acid sequence shown in SEQ ID NO:3 was most similar to the Lotus japonicus cytochrome P450 71 D11 having NCBI Accession No. 022307 and General Identifier No. 5915840. The BLASTP analysis showed a pLog of 148 and a 56% identity to the L. japonicus sequence.

### Example 3

### Expression of cytochrome P450s in Yeast and Assay for Production of 1-octen 3-ol

The ability of the cytochrome P450 encoded by the cDNA identified in Example 2 to produce 1-octen-3-ol was tested in yeast. Yeast expression vectors were prepared using the cDNA identified in Example 2 and transformed into yeast. Expression of the protein was accomplished by addition of galactose. Production of 1-octen-3-ol in yeast cultured in the presence or absence of galactose and linoleic acid was monitored using Stir Bar Sorptive Extraction (SBSE) techniques and was analyzed by gas chromatography mass spectrometry (GC-MS) as shown below.

Vector Construction

Vectors useful for expression in yeast were prepared with the cDNA insert of the clone identified in Example 2 as follows.

DNA was amplified from clone sfl1.pk0045.g7. The cDNA insert in clone sfl1.pk0045.g7 was amplified using Advantage HF2 polymerase (BD Biosciences, San Jose, Calif.) according to the manufacturer's instruction and 0.1 μL DNA. Primers PSO355954 sense-3 (SEQ ID NO:4) and PSO355954 anti-sense-3 (SEQ ID NO:5) were used to amplify nucleotides 22 through 1551 of the cDNA insert from clone sfl1.pk0045.g7

(SEQ ID NO:4)

5′-GCGGCCGCATGGCTCTATTATTCTTCTACTTTTTGG-3′

(SEQ ID NO:5)

5′-GCGGCCGCTCATGAGACAGGCAAAGGATGATATGG-3′

Amplification was performed using a GeneAmp PCR System 9700 machine (Applied Biosystems, Foster City, Calif.). Temperature cycles for amplification were: four minutes at 94° C.; followed by 30 cycles of 30 seconds at 94° C., 30 seconds at 53° C., and 60 seconds at 72° C.; followed by seven minutes at 72° C. The amplified DNA obtained from each reaction was cloned into PCR2.1 using the TOPO TA Cloning System (Invitrogen, Carlsbad, Calif.).

DNA from the plasmid prepared above was isolated using a Qiagen (Valencia, Calif.) miniprep kit according to the manufacturer's protocol and digested with Not I. DNA from the yeast expression vector pYES3/CT DNA (Invitrogen, Carlsbad, Calif.) was digested with Not I and treated with calf alkaline intestine phosphotase. DNA fragments were separated using a 1% TAE agarose gel, and the fragments of interest purified using a Qiagen gel purification kit. The cytochrome P450 DNA fragment was ligated with the prepared pYES3/CT DNA and transformed into E. coli.

Colonies were selected, grown under antibiotic selection, plasmid DNA from the resulting cultures was purified using a Qiagen miniprep kit according to the manufacturer's protocol, and analyzed by restriction digest. A plasmid containing the insert of interest was transformed into WHT1 yeast using the Saccharomyces cerevisiae EasyComp™ Transformation Kit (Invitrogen, Carlsbad, Calif.).

Yeast strain WHT1 was constructed essentially as described (Pompon, D., et al. (1996) Methods Enzymol. 272:51-64) but substituting the Helianthus tuberosum NADPH-ferrihemoprotein reductase (NCBI Accession No. Z26250) for the Arabidopsis thaliana NADPH-ferrihemoprotein reductase (Hasenfratz, M. P., 1992, Thesis, Universite Louis Pasteur, Strasbourg, France).

Yeast Growth and Analysis

Two strains of yeast, one containing the above described vector encoding a cytochrome P450 and a control strain containing pYES3/CT with no insert, were grown in a shaking incubator for 24 hours at approximately 26° C. The yeast were grown in synthetic complete media minus tryptophan with the addition of 0.2% (w/v) Tergitol NP-40 (Sigma, St. Louis, Mo.) in the presence or absence of 0.45 mM linoleic acid. Yeast cells were collected by centrifugation and the cell pellets resuspended in twice the original volume of media using media with or without galactose.

Galactose was used to induce expression of the cytochrome P450s because the P450 coding sequences are operably linked to the Gall promoter in plasmid pYES3/CT. The cultures were allowed to incubate for an additional 24 hours at approximately 26° C. SBSE techniques were used to collect the volatile products and deliver them to the Gas Chromatograph, and GC-MS was used to separate and identify the volatile products. Briefly, 1 cm×0.5 mm film thickness Twister® bars (Gerstel Inc, Baltimore, Md.) were placed into the yeast cultures to be analyzed. Volatile components from the media partition into the poly-dimethyl siloxane (PDMS) coating of the bars where they become concentrated. After a suitable incubation period (24 hours) the bars were removed from the media, washed thoroughly in deionized water, blotted dry, and loaded into sample tubes ready for introduction into the GC-MS. The GC-MS system comprised of an Agilent-6890 GC connected to an Agilent-5973 Mass Selective Detector (Agilent Technologies, Wilmington Del.). The Inlet of the GC was fitted with a Gerstel TDSA-autosampler and thermaldesorption apparatus coupled to a CIS4 programmed temperature vaporization (PTV) inlet. These components enable delivery, by thermaldesorption and cryo-focusing, of volatile components trapped on the Twister® bars, onto the chromatographic column. Chromatographic separation was performed on an Ultra-1, 50 m×0.32 mm (ID)×0.52 μm (film thickness) capillary column (Agilent Technologies, Wilmington Del.) with helium carrier gas at 30 cm/sec linear velocity. The temperature was held at 40° C. for 6 minutes, increased 4° C./minute to 270° C., increased 20° C./minute to 340° C., with a hold at the final temperature of 3.5 minutes. Compounds eluting from the column were detected with the Mass Spectrometer using electron impact fragmentation. Presence of 1-octen-3-ol was confirmed by observation of a compound with a mass spectrum that matched that of 1-octen-3-ol in the National Institute of Standards and Technology (NIST) Mass Spectral Library (Version 2.0 a), and with the same retention time and Retention Time Index (RTᵢ) value as an authentic sample of 1-octen-3-ol run under the same chromatographic conditions.

Incubations were performed with:

yeast transformed with a plasmid pYES3/CT containing cytochrome P450 clone sfl1.pk0045.g7, with addition of galactose and linoleic acid (at 0.45 mM).

yeast transformed with a plasmid pYES3/CT containing cytochrome P450 clone sfl1.pk0045.g7, with linoleic acid but without addition of galactose.

yeast transformed with a plasmid pYES3/CT containing cytochrome P450 clone sfl1.pk0045.g7, without addition of galactose or linoleic acid.

yeast transformed with plasmid pYES3/CT with no insert, with galactose and linoleic acid.

yeast transformed with plasmid pYES3/CT with no insert, with linoleic acid but without galactose.

yeast transformed with plasmid pYES3/CT with no insert, without galactose or linoleic acid.

The cultures containing yeast transformed with a plasmid containing cDNA derived from clone sfl1.pk0045.g7 produced 1-octen-3-ol when incubated in the presence of galactose and linoleic acid. This provides support that the cDNA insert in clone sfl1.pk0045.g7 encodes a cytochrome P450 that is involved in the production of 1-octen-3-ol.

### Example 4

### Isolation of a Genomic DNA Fragment

A genomic DNA fragment comprising the cDNA in clone sfl1.pk0045.g7 was obtained from soybean genomic DNA isolated from leaf tissue (cv. Jack) using the DNeasy plant DNA Maxi Kit (Qiagen, Valencia, Calif.) according to the manufacturer's protocol. One μL of DNA was amplified with primers gene 2 overexpress sense (SEQ ID NO:6) and gene 2 overexpress antisense (SEQ ID NO:7).

(SEQ ID NO:6)

5′-GCGGCCGCATGGCTCTATTATTCTTCTACTTTTTG-3′

(SEQ ID NO:7)

5′-GCGGCCGCTCATGAGACAGGCAAAGGATGATATGG-3′

Amplification was carried out using Advantage 2 polymerase (BD Biosciences, San Jose, Calif.) according to the manufacturer's instruction and using a GeneAmp PCR System 9700 machine (Applied Biosystems, Foster City, Calif.). Temperature cycles for amplification were: four minutes at 94° C.; followed by 30 cycles of 30 seconds at 94° C., 30 seconds at 55° C., and 2 minutes at 72° C.; followed by seven minutes at 72° C. The resulting DNA was cloned into PCR2.1 using the TOPO TA Cloning System (Invitrogen, Carlsbad, Calif.).

Colonies were selected, grown under antibiotic selection and DNA from the resulting cultures was purified using a Qiagen miniprep kit according to the manufacturer's protocol and analyzed by restriction digest analysis. DNAs were then sequenced using a combination of custom designed internal primers and plasmid-derived primers. The nucleotide sequence of a genomic fragment corresponding to the cDNA insert in clone sfl1.pk0045.g7 is shown in SEQ ID NO:8. Comparison of the nucleotide sequence of the genomic fragment with that of the cDNA insert in clone sfl1.pk0045.g7 indicates that translation of the genomic clone starts at nucleotide 15 (the “A” in the initial translation codon), is interrupted by an intron corresponding to nucleotides 921 through 1314, and nucleotides 1936 through 1938 are a termination codon. The amino acid sequence deduced from translating the coding sequence in the genomic fragment of SEQ ID NO:8 is shown in SEQ ID NO:22. This polypeptide contains a threonine (Thr) at amino acid 245 instead of the alanine (Ala) found in the amino acid sequence deduced from translating the nucleotide sequence of SEQ ID NO:2 and shown in SEQ ID NO:3.

### Example 5

### Preparation of Vectors for Transformation of Soybean (Glycine max) Embryos

Vectors useful for the overexpression or suppression of the polynucleotides of the invention may be prepared in many different ways. Below is a description of an overexpression vector and a suppression vector that have been prepared.

Vector pDN10

Plasmid pDN10 is an intermediate cloning vector comprising a bacterial origin of replication, bacterial and plant selectable marker gene expression cassettes, and a promoter and terminator separated by a unique Not I restriction endonuclease site. This plasmid was prepared by ligating a fragment comprising a plant selectable marker gene expression cassette and a cassette comprising a promoter and terminator separated by a unique Not I restriction endonuclease site to a fragment comprising the bacterial origin of replication and selectable marker gene. These two fragments were prepared as follows:

The first fragment has 6383 bp, was obtained by Kpn I digestion of pKS231, its nucleotide sequence is shown in SEQ ID NO:9, and contains two cassettes: 1) a plant selectable marker gene cassette, and 2) a cassette comprising a promoter and terminator separated by a unique Not I restriction endonuclease site. The plant selectable marker gene expression cassette comprises a 1.3-Kb DNA fragment that functions as the promoter for a soybean S-adenosylmethionine synthase (SAMS) gene directing expression of a mutant soybean acetolactate synthase (ALS) gene which is followed by the soybean ALS 3′ transcription terminator. The 1.3-Kb DNA fragment that functions as the promoter for a soybean SAMS gene has been described in PCT Publication No. WO 00/37662, published Jun. 29, 2000. The mutant soybean ALS gene encodes an enzyme that is resistant to inhibitors of ALS, such as sulfonylurea herbicides.

Mutant plant ALS genes encoding enzymes resistant to sulfonylurea herbicides are described in U.S. Pat. No. 5,013,659. One such mutant is the tobacco SURB-Hra gene, which encodes an herbicide-resistant ALS with two substitutions in the amino acid sequence of the protein. This tobacco herbicide-resistant ALS contains alanine instead of proline at position 191 in the conserved-“subsequence B” and leucine instead of tryptophan at position 568 in the conserved “subsequence F” (U.S. Pat. No. 5,013,659; Lee et al., 1988, EMBO J. 7:1241-1248).

The mutant soybean ALS gene was constructed using a polynucleotide for a soybean ALS to which the two Hra-like mutations were introduced by site directed mutagenesis. Thus, this recombinant DNA fragment will translate to a soybean ALS having alanine instead of proline at position 183 and leucine instead of tryptophan at position 560. The deduced amino acid sequence of the mutant soybean ALS present in the mutant ALS gene is shown in SEQ ID NO:10. During construction of SAMS promoter-mutant ALS expression cassette, the coding region of the soybean ALS gene was extended at the 5′ end by five additional codons, resulting in five amino acids, added to the amino-terminus of the ALS protein (amino acids 1 through 5 of SEQ ID NO:10). These extra amino acids are adjacent to and presumably removed with the transit peptide during targeting of the mutant soybean ALS protein to the plastid.

The cassette comprising a promoter and terminator separated by a unique Not I restriction endonuclease site comprises the KTi3 promoter, a unique Not I restriction endonuclease site, and the Kti3 terminator region. This cassette comprises about 2088 nucleotides of the Kti3 promoter, a unique Not I restriction endonuclease site, and about 202 nucleotides of the Kti3 transcription terminator. The gene encoding Kti3 has been described (Jofuku, K. D. and Goldberg, R. B., 1989, Plant Cell 1:1079-1093).

The second fragment, comprising the bacterial origin of replication and bacterial selectable marker gene was obtained by PCR amplification from plasmid pKS210 as follows. Plasmid pKS210 is derived from the commercially available cloning vector pSP72 (Promega, Madison, Wis.). To prepare plasmid pKS210 the beta lactamase coding region in vector pSP72 has been replaced by a hygromycin phosphotransferase (HPT) gene for use as a selectable marker in E. coli. The nucleotide sequence of plasmid pKS210 is shown in SEQ ID NO:11. A fragment of pKS210 comprising the bacterial origin of replication and the HPT gene was amplified by PCR using primers KS210-Kpn-Hyg-sense (SEQ ID NO:12) and KS210-Kpn-Hyg-antisense (SEQ IDNO:13), 0.25 micrograms of PKS210 as a template, and the Advantage High Fidelity polymerase (BD Biosciences, San Jose, Calif.) according to the manufacturer's instructions.

(SEQ ID NO:12)

GCCGGGGTACCGGCGCGCCCGATCATCCGGATATAGTTCC-3′

(SEQ ID NO:13)

GCCGGGGTACCGGCGCGCCGTTCTATAGTGTCACCTAATC-3′

A GeneAmp PCR System 9700 machine (Applied Biosystems, Foster City, Calif.) machine was used with the following temperature regime: 94° C. for 15 seconds and then 28 cycles of 94° C. for 15 seconds and 67° C. for 3 minutes followed by one cycle of 67° C. for 3 minutes. The resulting 2600 bp fragment was gel purified using the Qiagen Gel Purification System, digested with Kpn I and treated with Calf alkaline phosphatase.

The two Kpn I fragments described above were ligated together and transformed into E. coli. Bacterial colonies were selected and grown overnight in LB media and appropriate antibiotic selection. DNA was isolated from the resulting culture using a Qiagen Miniprep Kit according to the manufacturer's protocol and then analyzed by restriction digest. The resulting plasmid was named pDN 1 and its nucleotide sequence is shown in SEQ ID NO:14.

Preparation of Overexpression Vector

Plasmid PHP23366 was prepared to overexpress in plants the cytochrome P450 monooxygenase from clone sfl1.pk0045.g7. Plasmid PHP23366 comprises the open reading frame fragment in clone sfl1.pk0045.g7 inserted at the unique Not I site of plasmid pDN10. The open reading frame fragment in clone sfl1.pk0045.g7 was obtained by PCR amplification using primers to include Not I sites immediately upstream of the methionine-encoding ATG and immediately after the termination codon. Amplification was carried out using Advantage 2 polymerase (BD Biosciences, San Jose, Calif.) according to the manufacture's instruction, 0.1 μL of sfl1.pk0045.g7 DNA as the template, and primers gene 2 overexpress sense (SEQ ID NO:6) and gene 2 overexpress antisense (SEQ ID NO:7).

PCR amplification was performed using a GeneAmp PCR System 9700 machine (Applied Biosystems, Foster City, Calif.). Temperature cycles for amplification were: four minutes at 94° C.; followed by 30 cycles of 30 seconds at 94° C., 30 seconds at 55° C., and 60 seconds at 72° C.; followed by seven minutes at 72° C. The resulting DNA fragment was cloned into PCR2.1 using the TOPO TA Cloning System (Invitrogen, Carlsbad, Calif.), sequenced using a combination of custom designed internal primers and plasmid derived primers and shown to be identical to the template DNA with the exception of the addition of Not I sites.

DNA from the above plasmid was isolated using a Qiagen miniprep kit according to the manufacturer's protocol and then digested with Not I. Plasmid pDN10 DNA was digested with Not I and treated with Calf Alkaline Intestine Phosphatase. Both digestion reactions were loaded on 1% TAE agarose gels and the DNA fragments separated by electrophoresis. The fragments of interest were purified using a Qiagen gel purification kit. The purified fragment DNAs were ligated and transformed into E. coli.

Colonies were selected, grown under antibiotic selection and DNA from the resulting cultures was purified using a Qiagen miniprep kit according to the manufacturer's protocol and analyzed by restriction digest analyses. The resulting plasmid was named PHP23366. This plasmid comprises the open reading frame fragment in clone sfl1.pk0045.g7 inserted at the unique Not I site of plasmid pDN10 described above. Digestion of plasmid PHP23366 with Asc I results in Recombinant DNA Fragment PHP23366A (SEQ ID NO:17) which comprises cassettes for the expression of the open reading frame in clone sfl1.pk0045.g7 and the soybean mutant ALS gene operably linked to a promoter and terminator.

Preparation of a Suppression Vector

Plasmid PHP23367 was prepared to suppress in plants the cytochrome P450 monooxygenase from clone sfl1.pk0045.g7. Plasmid PHP23367 comprises a promoter operably linked to a fragment from the cDNA insert in clone sfl1.pk0045.g7 along with an inverted repeat of a portion of that same fragment.

Fragments comprising a portion of the cDNA insert in clone sfl1.pk0045.g7 were amplified using Advantage 2 polymerase (BD Biosciences, San Jose, Calif.) according to the manufacture's instruction. Amplification was performed with 0.1 μL DNA from clone sfl1.pk0045.g7 as template with primers gene 2 overexpress sense (SEQ ID NO:6) and gene 2 hairpin 2 (SEQ ID NO:1 5) or with primers gene 2 overexpress sense (SEQ ID NO:6) and gene 2 hairpin 3 (SEQ ID NO:16)

(SEQ ID NO:15)

CAAAGACCAAGAAAAATTTATACAACCTCAAGGTCTCTTTCAC

(SEQ ID NO:16)

TGAAAGAGACCTTGAGGTTGTATAAATTTTTCTTGGTCTTTG

PCR amplification was performed using a GeneAmp PCR System 9700 machine (Applied Biosystems, Foster City, Calif.). Temperature cycles for amplification were: four minutes at 94° C.; followed by 30 cycles of 30 seconds at 94° C., 30 seconds at 55° C., and 60 seconds at 72° C.; followed by seven minutes at 72° C.

An additional PCR amplification reaction was performed using 1 μL DNA from each of these two amplifications as template, using Advantage 2 polymerase (BD Biosciences, San Jose, Calif.) according to the manufacturer's instruction, and primer gene 2 overexpress sense (shown in SEQ ID NO:6). PCR amplification was performed using a GeneAmp PCR System 9700 machine (Applied Biosystems, Foster City, Calif.). Temperature cycles for amplification were: four minutes at 94° C.; followed by 30 cycles of 30 seconds at 94° C., 30 seconds at 55° C., and 90 seconds at 72° C.; followed by seven minutes at 72° C. The resulting DNA was cloned into PCR2.1 using the TOPO TA Cloning System (Invitrogen, Carlsbad, Calif.).

DNA from the above plasmid was isolated using a Qiagen miniprep kit according to the manufacturer's protocol and then digested with Not I. DNA from plasmid pDN10 was digested with Not I and treated with Calf Alkaline Intestine Phosphatase. These two reactions were separated by electrophoresis in a 1% TAE agarose gel. The resulting fragments were purified using a Qiagen gel purification kit and the purified DNA fragments were ligated and transformed into E. coli.

Colonies were selected, grown under antibiotic selection, and DNA from the resulting cultures was purified using a Qiagen miniprep kit according to the manufacturer's protocol and analyzed by restriction digest analyses. The resulting plasmid was named PHP23367. Digestion of PHP23367 with Asc I results in a 8121 bp recombinant DNA fragment called PHP23367A (SEQ ID NO:18) that comprises a promoter operably linked to fragments of sfl1.pk0045.g7 designed to form a hairpin structure followed by a terminator as well as the soybean mutant ALS gene operably linked to a promoter and terminator. For use in plant transformation experiments the 8121 bp recombinant DNA fragment PHP23367A was purified by agarose gel electrophoresis.

### Example 6

### Transformation of Somatic Soybean (Glycine max) Embryos and Regeneration of Soybean Plants

Soybean products prepared from plants producing less 1-octen-3-ol are expected to be more palatable to humans. Thus, transgenic plants comprising a portion of a cytochrome P450 monooxygenase capable of suppressing production of 1-octen-3-ol were produced.

Soybean embryogenic suspension cultures were transformed by the method of particle gun bombardment using procedures known in the art (Klein, T., et al. 1987, Nature (London) 327:70-73; U.S. Pat. No. 4,945,050; Hazel, C. B., et al., 1998, Plant Cell. Rep. 17:765-772; Samoylov, et al., 1998, In Vitro Cell Dev. Biol. Plant 34:8-13). In particle gun bombardment procedures it is possible to use purified either entire plasmid DNA or DNA fragments containing only the recombinant DNA expression cassette(s) of interest.

In the Example that follows, the recombinant DNA fragment PHP23367A was isolated from the plasmid PHP23367 by Acc I digestion and gel electrophoresis before being used for bombardment. For every eight bombardment transformations, 30 mL of solution were prepared with 3 mg of 0.6 mm gold particles and 1 to 90 picograms (pg) of DNA fragment per base pair of DNA fragment.

Stock tissue for these transformation experiments was obtained by initiation from soybean immature seeds. Secondary embryos were excised from explants after 6 to 8 weeks on culture initiation medium. The initiation medium was an agar-solidified modified MS medium (Murashige and Skoog, 1962, Physiol. Plant. 15:473497) supplemented with vitamins, 2,4-D, and glucose. Secondary embryos were placed in flasks in liquid culture maintenance medium and maintained for 7-9 days on a gyratory shaker at 26 +/−2° C. under ˜80 μEm-2s-1 light intensity. The culture maintenance medium was a modified MS medium supplemented with vitamins, 2,4-D, sucrose, and asparagine. Prior to bombardment, clumps of tissue were removed from the flasks and moved to an empty 60×15 mm petri dish for bombardment. Tissue was dried by blotting on Whatman #2 filter paper. Approximately 100-200 mg of tissue corresponding to 10-20 clumps (1-5 mm in size each) were used per plate of bombarded tissue.

After bombardment, tissue from each bombarded plate was divided and placed into two flasks of liquid culture maintenance medium per plate of bombarded tissue. Seven days post bombardment the liquid medium in each flask was replaced with fresh culture maintenance medium supplemented with 100 ng/mL selective agent (selection medium). For selection of transformed soybean cells the selective agent used was a sulfonylurea (SU) compound with the chemical name, 2-chloro-N-((4-methoxy-6 methy-1,3,5-triazine-2-yl)aminocarbonyl) benzenesulfonamide (common names: DPX-W4189 and chlorsulfuron). Chlorsulfuron is the active ingredient in the DuPont sulfonylurea herbicide, GLEAN®. The selection medium containing SU was replaced every week for 6-8 weeks. After the 6-8 week selection period, islands of green, transformed tissue were observed growing from untransformed, necrotic embryogenic clusters. These putative transgenic events were isolated and kept in media with SU at 100 ng/mL for another 2-6 weeks with media changes every 1-2 weeks to generate new, clonally propagated, transformed embryogenic suspension cultures. Embryos spent a total of around 8-12 weeks in SU. Suspension cultures were subcultured and maintained as clusters of immature embryos and also regenerated into whole plants by maturation and germination of individual somatic embryos.

Production of 1-octen-3-ol will be monitored by GC-MS or GC-Flame ionization Detector (FID) analysis of seeds from plants containing the transgene and from plants not containing the transgene. These analyses will be performed following the procedures outlined in Example 3.

### Example 7

### Identification of Mutations in Novel Cytochrome P450 Monooxygenases

The availability of polynucleotides encoding the cytochrome P450s of the invention makes it straightforward to identify mutations in the endogenous genes using a variety of methods well known to those skilled in the art. For example, a strategy called TILLING (for Targeting Induced Local Lesions in Genomes) can be used for the introduction and identification of mutations in the cytochrome P450s of the invention. In TILLING traditional chemical mutagenesis is followed by high-throughput screening for point mutations (Henikoff, S. et al., 2004, Plant Phys. 135: 1-7). Seeds are mutagenized by treatment with ethylmethanesulfonate (EMS). The resulting M1 plants are self-fertilized, and M2 individuals are used to prepare DNA samples for mutational screening. The DNA samples are pooled and arrayed in microtiter plates, and the pools are amplified using gene-specific primers such as those disclosed in SEQ ID NOs: 4, 5, 6, and 7. Amplification products are incubated with the CEL I endonuclease, a member of the S1 nuclease family of single strand-specific nucleases (Oleykowski, C. A. et al., 1998, Nucleic Acids Res 26: 4597-4602). CEL I cleaves to the 3′ side of mismatches and loop outs in heteroduplexes between wild-type and mutant DNA while leaving duplexes intact. Cleavage products are separated by electroporation using the LI-COR (Lincoln, Nebr.; Middendorf et al., 1992, Electrophoresis 13: 487-494) gel analyzer system, and a standard commercial image processing program (e.g., Adobe Photoshop; Adobe Systems, Mountain View, Calif.) is used to examine the gel read-out. Differential double-end labeling of amplification products allows for rapid visual confirmation because mutations are detected on complementary strands and so can be easily distinguished from amplification artifacts. Upon detection of a mutation in a pool, the individual DNA samples are similarly screened to identify the individual carrying the mutation. This rapid screening procedure determines the location of a mutation to within 610 bp for PCR products that are 1-kb in size.

Similarly, the availability of polynucleotides encoding the cytochrome P450s of the invention makes it possible to identify transposon insertion mutations that affect their expression by comparison to the genomic sequence, SEQ ID NO: 8. First a library of soybean transposon insertion mutations must be generated. A method to obtain Agrobacterium T-DNA insertions in soybean has been described (Olhlft et al., 2004, Plant Biotech. J. 2: 289-300), and could be used to generate such a library. Then, a method for rapid identification of T-DNA insertion mutants could be used to identify cytochrome P450 mutants (Rios et al., 2002, Plant J. 32: 243-253). PCR primers, which are designed based on the gene sequence, are used with a sensitive PCR technique to permit identification of T-DNA tagged genes without DNA hybridization. The PCR screening is performed by agarose gel electrophoresis followed by isolation and direct sequencing of DNA fragments of amplified T-DNA insert junctions. The precise location of a transposon insertion in the gene can be determined by sequencing of genomic DNA from individual mutants. Because both TILLING and transposon mutation screening do not involve transgenic modifications, they are attractive for agricultural applications.

Production of 1-octen-3-ol in seeds from mutant plants carrying at least one mutation in the cytochrome P450 of the invention is then compared to that of non-mutant plants using GC-MS or GC-FID as described in Example 3.

### Example 8

### Large Scale Production of 1-octen-3-ol

Large-scale production of 1-octen-3-ol using a recombinant yeast host may be accomplished in a Fed-batch system or continuous culture.

Fed-batch culture processes are suitable in the present invention and comprise a typical batch system with the exception that the substrate is added in increments as the culture progresses. A classical batch culturing method is a closed system where the composition of the media is set at the beginning of the culture and not subject to external alterations during the culturing process. Thus, at the beginning of the culturing process the media is inoculated with the desired organism or organisms and growth or metabolic activity is permitted to occur while adding nothing to the system. Batch and Fed-batch culturing methods are common and well known in the art and examples may be found in several references including Thomas D. Brock, 1989, in Biotechnology: A Textbook of Industrial Microbiology, 2^{nd} ed., Sinauer Associates: Sunderland, Mass., or Deshpande, M. V., 1992, Appl. Biochem. Biotechnol. 36:227-234.

Commercial production of a product of interest in yeast may also be accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth.

Continuous or semi-continuous culture allows for the modulation of one factor or any number of factors that affect cell growth or end product concentration. For example, one method will maintain a limiting nutrient such as the carbon source or nitrogen level at a fixed rate. In other systems a number of factors affecting growth can be altered continuously while the cell concentration, measured by media turbidity, is kept constant. Production of 1-octen-3-ol may use this method to add linoleic acid as needed. Continuous systems strive to maintain steady state growth conditions and thus the cell loss due to media being drawn off must be balanced against the cell growth rate in the culture. Methods of modulating nutrients and growth factors for continuous culture processes, as well as techniques for maximizing the rate of product formation, are well known in the art of industrial microbiology and a variety of methods are detailed by Brock, supra.

Scale up of the method used to express the polynucleotides of the invention in Example 3 may be used. Several scale up methods and approaches are described by Junker, B. H. (2004, J. Biosci. Bioeng. 97:347-364).

### Example 9

### Isolation of P450 Monooxygenase Promoter

The promoter of a soybean P450 monooxygenase was isolated using a polymerase chain reaction (PCR) based approach. Soybean libraries were prepared and amplification was carried out using the GenomeWalker Kit™ (Clonetech, Palo Alto, Calif.) according to the manufacturer's instructions. The GenomeWalker Kit™ allows PCR-based, bidirectional DNA walking from known cDNA sequences without screening a library. Two gene-specific primers were designed based on the nucleotide sequence of the cDNA insert in clone sfl1.pk0045.g7 whose sequence is shown in SEQ ID NO:2. The gene-specific primers designed were gene specific primer 1 and gene specific primer 2. The nucleotide sequence of gene specific primer 1 and gene specific primer 2 are shown in SEQ ID NO:19 and SEQ ID NO:20, respectively, and have the sequences shown as follows:

| | |
|---|---|
| 5′-TGGCCAGGTCTCTTAATTTTCTATGG-3′ | (SEQ ID NO:19) |
| 5′-AGGACCATGGGGTATTTTACAAGTTG-3′ | (SEQ ID NO:20) |

Amplification was performed using a GeneAmp PCR System 9700 machine (Applied Biosystems, Foster City, Calif.) using ExTaq (TaKaRa, Japan), again according to the manufacturer's instructions. Several putative promoter fragments were obtained and cloned into PCR2.1 using the TOPO TA Cloning System (Invitrogen, Carlsbad, Calif.). Portions of the putative promoter fragments were sequenced and the sequences obtained compared to those of SEQ ID NO:2. The longest fragment whose sequence overlapped that of SEQ ID NO:2 was completely sequenced in both directions using a combination of external plasmid primers and custom internal primers and its sequence is shown in SEQ ID NO:21. The ATG that is the start of translation is at position 2654-2656.

What is claimed is:

1. An isolated polynucleotide comprising:
(a) a nucleotide sequence encoding a cytochrome P450 polypeptide having an amino acid sequence of at least 80% sequence identity, based on the Clustal V method of alignment, when compared to an amino acid sequence of SEQ ID NO:3 , wherein expression of said polypeptide in an appropriate host cell transformed with said isolated polynucleotide, in the presence of linoleic acid, results in an increased level of 1-octen-3-ol in said transformed host cell when compared to an nontransformed host cell; or
(b) a complement of the nucleotide sequence, wherein the complement and the nucleotide sequence consist of the same number of nucleotides and are 100% complementary.

2. A portion of the polynucleotide of claim 1 wherein said portion is capable of suppressing production of 1-octen-3-ol when introduced into cells that normally produce 1-octen-3-ol.

3. A portion of SEQ ID NOS: 2, 8, or 21 wherein said portion is capable of suppressing production of 1-octen-3-ol when introduced into cells that normally produce 1-octen-3-ol.

4. A polynucleotide of claim 1 wherein said nucleotide sequence further comprises a native cytochrome P450 promoter region, a native cytochrome P450 terminator region, and a native cytochrome P450 intron.

5. The polynucleotide of claim 4 wherein the nucleotide sequence comprises the nucleotide sequence in SEQ ID NO:8.

6. The polynucleotide of claim 4 wherein the native cytochrome P450 promoter region comprises the nucleotide sequence in SEQ ID NO:21.

7. The isolated polynucleotide of claim 1, wherein said polypeptide has an amino acid sequence of at least 85% sequence identity, based on the Clustal V method of alignment, when compared to an amino acid sequence of SEQ ID NO:3.

8. The isolated polynucleotide of claim 1, wherein said polypeptide has an amino acid sequence of at least 90% sequence identity, based on the Clustal V method of alignment, when compared to an amino acid sequence of SEQ ID NO:3.

9. The isolated polynucleotide of claim 1, wherein said polypeptide has an amino acid sequence of at least 95% sequence identity, based on the Clustal V method of alignment, when compared to an amino acid sequence of SEQ ID NO:3.

10. The isolated polynucleotide of claim 1, wherein said polypeptide has an amino acid sequence of SEQ ID NO:3.

11. The isolated polynucleotide of claim 1, wherein said polypeptide has an amino acid sequence of SEQ ID NO:22.

12. A recombinant DNA construct comprising the polynucleotide of claims 1, 2, 3, 4, 5, 6, 7, 8, 9, 10, or 11 operably linked to at least one regulatory sequence.

13. A transgenic cell comprising the recombinant DNA construct of claim 12.

14. The cell of claim 13 wherein expression of said recombinant DNA construct results in an altered level of 1-octen-3-ol.

15. The cell of claim 13 wherein expression of said recombinant DNA construct results in an increase in 1-octen-3-ol.

16. The cell of claim 13 wherein expression of said recombinant DNA construct results in a decrease in 1-octen-3-ol.

17. A plant comprising the recombinant DNA construct of claim 12.

18. A plant transformed with the recombinant DNA construct of claim 12 and having an altered level of 1-octen-3-ol when compared to a non-transformed plant.

19. A plant transformed with the recombinant DNA construct of claim 12 and having an increased level of 1-octen-3-ol when compared to a non-transformed plant.

20. A plant transformed with the recombinant DNA construct of claim 12 and having a decreased level of 1-octen-3-ol when compared to a non-transformed plant.

21. A soybean plant whose genome comprises a disruption of the polynucleotide of claims 1, 2, 3, 4, 5, 6, 7, 8, 9, 10 or 11 wherein said disruption results in said plant exhibiting reduced 1-octen-3-ol when compared to its wild type counterpart.

22. The plant of claims 17, 18, 19, or 20, wherein said plant is a soybean plant.

23. A seed comprising the recombinant DNA construct of claim 12.

24. The seed of claim 23 wherein said seed is a soybean seed.

25. A method for transforming a cell, comprising transforming a cell with the recombinant DNA construct of claim 12.

26. A method for producing a plant comprising:
transforming a plant cell with the recombinant DNA construct of claim 12,
regenerating a plant from the transformed plant cell; and
growing the transformed plant under conditions suitable for the expression of the recombinant DNA construct, said grown transformed plant having an altered level of 1-octen-3-ol when compared to a non-transformed plant.

27. The method of claim 26, wherein said plant is a soybean plant.

28. The method of claim 26, wherein said grown transformed plant has an increased level of 1-octen-3-ol when compared to a non-transformed plant.

29. The method of claim 26, wherein said grown transformed plant has a decreased level of 1-octen-3-ol when compared to a non-transformed plant.

30. A method of producing 1-octen-3-ol comprising: transforming a host cell with the recombinant DNA construct of claim 12; and adding linoleic acid to said transformed host cell in an amount sufficient for said host cell to produce 1-octen-3-ol.

31. The method of claim 30 wherein said host cell is selected from the group consisting of yeast and an insect cell.

32. Soybean grain from the plant of claim 22.

33. Soybean protein product prepared from the soybean grain of claim 32.

34. Soybean oil prepared from the soybean grain of claim 32.

35. Feed prepared from the soybean grain of claim 32.

36. A food prepared from the soybean grain of claim 32.

37. A food prepared with the soybean protein product of claim 33.
